SEQUENCE LISTING (16 sequences)

SEQ ID NO: 1 - protein, 683 aa, Murine Leukaemia Virus
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPNSRLIN

SEQ ID NO: 2 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAATAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPNSRLIN

SEQ ID NO: 3 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVQYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPNSRLIN

SEQ ID NO: 4 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIEDSSPNSRLIN

SEQ ID NO: 5 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWARLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPNSRLIN

SEQ ID NO: 6 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVQYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAATAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPNSRLIN

SEQ ID NO: 7 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAATAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIEDSSPNSRLIN

SEQ ID NO: 8 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWARLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAATAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPNSRLIN

SEQ ID NO: 9 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVQYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIEDSSPNSRLIN

SEQ ID NO: 10 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWARLPQGFKNSPTLFDEALHRDLADFRIQHPDLILLQYVDDLLLAATSELDCQQGTRALLQTLGNLGYRASAKKAQICQKQVQYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPNSRLIN

SEQ ID NO: 11 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWARLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIEDSSPNSRLIN

SEQ ID NO: 12 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVQYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAATAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIEDSSPNSRLIN

SEQ ID NO: 13 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWARLPQGFKNSPTLFDEALHRDLADFRIQHPDLILLQYVDDLLLAATSELDCQQGTRALLQTLGNLGYRASAKKAQICQKQVQYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAATAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPNSRLIN

SEQ ID NO: 14 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWARLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAATAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIEDSSPNSRLIN

SEQ ID NO: 15 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWARLPQGFKNSPTLFDEALHRDLADFRIQHPDLILLQYVDDLLLAATSELDCQQGTRALLQTLGNLGYRASAKKAQICQKQVQYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIEDSSPNSRLIN

SEQ ID NO: 16 - protein, 683 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWARLPQGFKNSPTLFDEALHRDLADFRIQHPDLILLQYVDDLLLAATSELDCQQGTRALLQTLGNLGYRASAKKAQICQKQVQYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAATAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIEDSSPNSRLIN

(19) United States
(12) Patent Application Publication
(10) Pub. No.: US 2026/0308541 A1
(43) Pub. Date: Oct. 8, 2026

(54) REVERSE TRANSCRIPTASE WITH IMPROVED PROPERTIES

(71) Applicant: Thermo Fisher Scientific Baltics UAB, Vilnius (LT)

(72) Inventors: Renata Rimseliene, Vilnius (LT); Jonas Virkutis, Vilnius (LT); Marija Zvirbliene, Vilnius (LT)

(21) Appl. No.: 19/483,494

(22) PCT Filed: May 21, 2024
(86) PCT No.: PCT/EP2024/064000

§ 371 Date: Nov. 11, 2025

(30) Foreign Application Priority Data
May 22, 2023 (GB) 2307629.2

(51) Int. Cl. C12N 9/12; C12P 19/34

(52) U.S. Cl. CPC C12N 9/1276; C12P 19/34; C12Y 207/07049

(57) ABSTRACT

The present invention relates to reverse transcriptase enzymes comprising a modified Moloney Murine Leukaemia Virus reverse transcriptase amino acid sequence, wherein said amino acid sequence comprises a mutation compared to the wild-type Moloney Murine Leukaemia Virus reverse transcriptase, and said enzyme possesses increased inhibitor resistance compared to the wild-type Moloney Murine Leukaemia Virus reverse transcriptase. The present invention also relates to compositions, reaction mixtures, and kits comprising the said reverse transcriptase enzymes are used to synthesize cDNA from RNA.

### BACKGROUND

The present invention relates to reverse transcriptases with improved properties. Reverse transcriptases are RNA-dependent DNA polymerases that synthesise DNA using RNA as a template. Said synthesis of DNA using RNA as a template is routinely followed by polymerase chain reaction (PCR) to rapidly detect and quantify the obtained complementary DNA (cDNA). Therefore, reverse transcriptases are essential reagents used in molecular biology applications.

Commonly used RTs are derived from Moloney Murine Leukaemia Virus (MMLV) and Avian Myeloblastosis Virus (AMV). While these derivatives possess certain advantages, they are often not sufficiently resistant to inhibitors that may be inherently present in some samples, sometimes even after clean-up of the samples. Improved inhibitor resistance is particularly important in one-step RT-quantitative PCR (qPCR) and one step RT-PCR in which both cDNA synthesis and qPCR are performed in the same reaction vessel. Another important area where improved inhibitor resistance is needed is direct RT-PCR. Hence, the newly synthesised cDNA is prone to contamination with inhibitors from its source, for example blood or soil.

Furthermore, one-step RT-qPCR is preferably used in high-throughput applications and diagnostics since one-step RT-qPCR translates into reduced sample handling, thereby reducing the risk of cross-contamination and experimental errors. Consequently, it is desirable to further reduce the bench time and therefore, to increase the time efficiency of one-step RT-qPCR by providing reverse transcriptases that do not require additional repeat experiments with further clean-up steps.

In some instances, the disclosed reverse transcriptases comprise additional desirable properties such as, for example, increased thermoactivity, increased thermostability, increased processivity and/or increased cDNA synthesis speed compared to wild-type MMLV reverse transcriptases.

Therefore, this invention provides improved reverse transcriptases that are based on MMLV reverse transcriptase and confer at least improved inhibitor resistance.

### SUMMARY OF THE INVENTION

In one aspect, the invention provides a reverse transcriptase enzyme comprising a modified Moloney Murine Leukaemia Virus (MMLV) reverse transcriptase amino acid sequence. Said amino acid sequence comprises a mutation at position I416, and/or K267, and/or N674, and/or T186 compared to the wild-type MMLV reverse transcriptase of SEQ ID NO: 1, and said enzyme possesses increased inhibitor resistance compared to said wild-type MMLV reverse transcriptase.

In one embodiment, the reverse transcriptase enzyme has at least two mutations at positions selected from the group consisting of the following: I416, K267, N674 and T186.

In another embodiment, the reverse transcriptase enzyme has at least two mutations at positions I416 and K267.

In yet another embodiment, the reverse transcriptase enzyme has at least two mutations at positions N674 and K267.

In one embodiment, the reverse transcriptase enzyme comprises at least one further mutation at one or more of the following amino acid positions:

| | | | | |
|---|---|---|---|---|
| P51 | H204 | N249 | T306 | F309 |
| D524 | E562 | K571 | D583 | |
| T330 | N479 | L603 | M289 | |
| M66 | L139 | D200 | T287 | H594 |
| E607 | Q221 | I49 | A502 | D653 |
| K658 | P130 | Q237 | A307 | Y344 |
| Q430 | D449 | A644 | N649 | L671 |
| E673 | M39 | Q91 | W388 | I179 |
| L333 | R390 | Q374 | E5 | |
| E69 | E302 | W313 | L435 | N454 |
| D124 | E286 | | | |

In one embodiment, the reverse transcriptase enzyme comprises a mutation selected from the group consisting of I416Q, I416N, I416T, I416S. In a preferred embodiment, the reverse transcriptase enzyme comprises an I416T mutation.

In another embodiment, the reverse transcriptase enzyme comprises a mutation at position K267 that is not K267T. In a preferred embodiment, the reverse transcriptase enzyme comprises a mutation selected from the group consisting of K267Q, K267N, K267S. In a particularly preferred embodiment, the reverse transcriptase enzyme comprises a K267Q mutation.

In yet another embodiment, the reverse transcriptase enzyme comprises a mutation selected from the group consisting of N674D or N674E. In a preferred embodiment, the reverse transcriptase enzyme comprises a N674D mutation.

In even another embodiment, the reverse transcriptase enzyme comprises a mutation at position T186 that is not T186C or T186D. In a preferred embodiment, the reverse transcriptase enzyme comprises a mutation selected from the group consisting of T186A, T186V, T186I, T186L, T186M.

In a particularly preferred embodiment, the reverse transcriptase enzyme comprises a T186A mutation.

In one embodiment, the reverse transcriptase enzyme comprises an I416T mutation, a K267Q mutation, a N674D mutation, and/or a T186A mutation. In a preferred embodiment, reverse transcriptase enzyme comprises an I416T mutation, a K267Q mutation, and/or a N674D mutation.

In another preferred embodiment, the reverse transcriptase enzyme comprises an I416T mutation and a K267Q mutation.

In another embodiment, the reverse transcriptase enzyme confers increased resistance to hemin, tannic acid, humic acid, and guanidine isothiocyanate.

In another aspect, the invention provides a composition comprising the reverse transcriptase according to any of the preceding embodiments.

In another aspect, the invention provides a reaction mixture comprising the reverse transcriptase according to any of the preceding embodiments.

Another aspect of the invention is the use of the reverse transcriptase according to any of the preceding embodiment for reverse transcription of RNA.

In yet another aspect, the invention provides a kit comprising the reverse transcriptase according to any of the preceding embodiments.

In even another aspect, the invention provides a polynucleotide encoding the reverse transcriptase enzyme according to any preceding embodiment. In one embodiment, the polynucleotide is codon-optimised for expression in a target host.

In one aspect, the invention provides a plasmid comprising the polynucleotide according to the preceding aspect and embodiment.

In another aspect, the invention provides a cell comprising the reverse transcriptase according to any one of the preceding embodiments or the polynucleotide of the preceding aspect and associated embodiment.

In yet another aspect, the invention provides a method for reverse transcription of RNA. In one embodiment, the reverse transcriptase according to any of the preceding embodiments is used to synthesise cDNA from RNA in a sample. In another embodiment, the sample is selected from the group consisting of a biological sample, environmental sample, food product sample. In a preferred embodiment, the biological sample, environmental sample, or food product sample is a processed sample or a purified sample. In an equally preferred embodiment, the biological sample, environmental sample, or food product sample is an unprocessed sample or an unpurified sample.

### Definitions

“cDNA synthesis speed” refers to the ability of a reverse transcriptase to synthesise certain amounts of cDNA in a certain time period, under suitable reaction conditions. Increased speed may relate to increased affinity to the template, increased processivity, increased nucleotide incorporation rate etc.

“Inhibitor resistance” refers to the ability of a reverse transcriptase to perform reverse transcription in the presence of a compound, chemical, protein, buffer, etc. that is typically inhibitory to the reverse transcriptase (prevents or inhibits reverse transcriptase activity). Exemplary compounds or chemicals are, for example, detergents, anti-coagulants or polysaccharides. In particular, the reverse transcriptase according to the invention confers increased resistance to hemin, tannic acid, humic acid, and guanidine isothiocyanate. More specifically, the reverse transcriptase according to the invention demonstrates increased resistance compared to wild-type MMLV reverse transcriptase (SEQ ID NO: 1), such that the inventive reverse transcriptase is capable of reverse transcription in the presence of up to 15 μM hemin, up to 7.5 ng/μL tannic acid, up to 10 ng/μL humic acid and up to 80 mM guanidine isothiocyanate. The reverse transcriptase according to the invention confers increased resistance to heparin, xylan, isopropanol and ELUGENT™ detergent, too.

“Processivity” of a reverse transcriptase refers to the number of nucleotides incorporated in a single binding event of the enzyme. Therefore, a highly processive reverse transcriptase can synthesize longer cDNA strands in a shorter reaction time. Enzyme processivity is also associated with affinity for the template.

“Sample” refers to a sample or specimen from biological, environmental or food product sources. Biological, environmental and food product samples contain a nucleic acid of interest that can be detected and quantified. The samples can be fresh samples, frozen samples, or preserved samples, for example, preserved in formalin. The samples according to the invention can contain further compounds such as metabolites of drugs, antibiotics, anticoagulants, chemicals such as preservatives, fixatives or buffers, nutrients, fertilisers or the like. In some instances, the sample is from a contaminated source.

Biological samples refer to a sample from a subject that can be an animal such as a mammal or human. Said sample or specimen may be solid, fluid, a tissue, or cells. Biological sample also refers to a culture such as a cell culture or microbiological culture. All biological fluids and excretions are included.

Environmental samples concern both samples from nature, such as soil or water, and the surroundings, including the environment or surroundings which are not nature, such as the interior of buildings such as walls, floors, surfaces, ventilators, drains, conveyor belts, and containers.

Food product samples are samples from food or beverage sources at any stage. The sample can be a raw material, a material that is being processed, an “in-process sample”, or a sample from the finalised food product.

A processed sample refers to a sample that has been subjected to a defined process or treatment. Said defined process or treatment may comprise multiple steps. In some instances, a purified sample (see definition below) is subjected to a defined process or treatment, thereby becoming a processed sample.

A purified sample refers to a sample from which any substance that is potentially harmful, dirty or inferior has been removed. In other words, substances considered to be contaminants are removed from a purified sample. In some instances, a purified sample is a processed sample.

“Target host” refers to any suitable organism or cellular subunit thereof that can harbour the plasmid or polynucleotide comprising the reverse transcriptase according to the invention. Therefore, a suitable organism can be, for example, a yeast cell, a prokaryotic cell, or eukaryotic cells or cell lines. In some instances, the target host can express and therefore produce the reverse transcriptase according to the invention.

“Thermoactivity” or “thermoactive” refers generally to the ability of a reverse transcriptase to exhibit enzyme activity at elevated temperatures.

“Thermostability” or “thermostable” refers generally to the ability to withstand exposure to elevated temperatures, but not necessarily show activity at such elevated temperatures.

“Composition” refers generally to a product comprising various components. Here, a composition comprises the inventive reverse transcriptase. The composition can further comprise salts. In one particular example, the composition comprises Tris-HCl, KCl, and MgCl₂. The composition can further comprise actinomycin D, ribonucleotides, and/or dithiothreitol.

“Reaction mixture” refers generally to a mixture comprising two or more components that cause (a) reaction(s) of the original two or more components. For example, the reaction mixture can be a RT-qPCR or RT-PCR reaction mixture. Such reaction mixtures typically comprise the RNA of interest, a reverse transcriptase, a reverse transcriptase buffer, a RNase inhibitor, ribonucleotides, one or more primers and nuclease-free water. The reaction mixtures can further comprise additives known to facilitate or enhance reverse transcription, amplification, or a combination of both reactions (e.g., agents for facilitating or enhancing RT-PCR) Suitable additives are listed in the detailed description below. The reaction mixture can also further comprise one or more surfactants and/or detergents to support stability upon storage that are listed in the detailed description below. Additionally, the reaction mixture can further comprise one or more dyes or fluorophores that are listed in the detailed description below, too.

“Stable” and “stability” in the context of compositions refer to the retention by a composition, such as an enzyme composition, of at least 70%, preferably at least 80%, and most preferably at least 90%, of the original enzymatic activity (in units) after the enzyme or composition containing the enzyme has been stored for about one week at a temperature of about 4° C., about two to six months at a temperature of about −20° C., and about six months or longer at a temperature of about −80° C.

“Working concentration” means the concentration of an enzyme that is at or near the optimal concentration used in a solution to perform a particular function such as reverse transcription of nucleic acids.

### FIGURES

FIG. 1. Synthesis of 1.3 kb cDNA fragment from GAPDH RNA with wild-type MMLV reverse transcriptase or its variants containing single amino acid substitution indicated above the picture. The reaction mixtures contained increasing amounts of hemin. The numbers above the picture indicate hemin concentrations (μM) in each reaction mixture. L—ZipRuler Express 1 DNA Ladder (ID 1373, Thermo Scientific).

FIG. 2. Resistance to inhibitors of the single point mutants compared to wild-type MMLV reverse transcriptase. Colours indicate the amount of 1.3 kb cDNA fragment obtained during reverse transcription reactions: white—the amount of the product (1.3 kb cDNA fragment) is identical to negative control (reaction mixture without inhibitor); light grey—decreased amount of product, dark grey—no detectable product.

FIG. 3. Synthesis of 0.5-9 kb cDNA fragments from Millennium™ RNA Markers (Invitrogen, #AM7150) with single point mutants I416T (SEQ ID NO: 2), K267Q (SEQ ID NO: 3), N674D (SEQ ID NO: 4) and T186A (SEQ ID NO: 5) compared to wild-type MMLV reverse transcriptase (SEQ ID NO: 1). Reaction products were analysed in 1.4% alkaline agarose gel.

FIG. 4. Thermostability and thermoactivity of the single point mutant I416T (SEQ ID NO: 2) compared to wild-type MMLV reverse transcriptase (SEQ ID NO: 1). 0.5-9 kb cDNA fragments from Millennium™ RNA Markers (Invitrogen, #AM7150) were synthesized at different temperatures as indicated above the picture (42° C., 44° C., 46° C., 48° C., 50° C., respectively). Reaction products were analysed in 1.0% alkaline agarose gel.

### DETAILED DESCRIPTION

### Reverse Transcriptase Enzyme

In one aspect, the invention provides a reverse transcriptase enzyme comprising a modified Moloney Murine Leukaemia Virus (MMLV) reverse transcriptase amino acid sequence. Said amino acid sequence comprises a mutation at position I416, and/or K267, and/or N674, and/or T186 compared to wild-type MMLV reverse transcriptase (SEQ ID NO: 1), wherein said enzyme possesses increased inhibitor resistance compared to said wild-type MMLV reverse transcriptase.

In one embodiment, the reverse transcriptase enzyme comprising a modified MMLV reverse transcriptase amino acid sequence is substantially identical or substantially homologous to SEQ ID NOs: 2, 3, 4, or 5. In another embodiment, the reverse transcriptase enzyme comprising a modified MMLV reverse transcriptase amino acid sequence is substantially identical or substantially homologous to SEQ ID NOs: 2. In even another embodiment, the reverse transcriptase enzyme comprising a modified MMLV reverse transcriptase amino acid sequence is substantially identical or substantially homologous to SEQ ID NOs: 3. In yet another embodiment, the reverse transcriptase enzyme comprising a modified MMLV reverse transcriptase amino acid sequence is substantially identical or substantially homologous to SEQ ID NOs: 4. In one embodiment, the reverse transcriptase enzyme comprising a modified MMLV reverse transcriptase amino acid sequence is substantially identical or substantially homologous to SEQ ID NOs: 5. In one preferred embodiment, the reverse transcriptase enzyme comprising a modified MMLV reverse transcriptase amino acid sequence shares at least 50%, 60%, 70%, 75%, 80%, 85%, 90%, 91%, 92%, 93%, 94%, 95%, 96%, 97%, 98%, or 99%, or more sequence identity with SEQ ID NOs: 2, 3, 4, or 5. In a particularly preferred embodiment, the reverse transcriptase enzyme comprising a modified MMLV reverse transcriptase amino acid sequence shares at least 95%, 96%, 97%, 98%, or 99%, or more sequence identity with SEQ ID NOs: 2, 3, 4, or 5.

In another embodiment, the reverse transcriptase enzyme comprising a modified MMLV reverse transcriptase amino acid sequence is substantially identical or substantially homologous to wild-type MMLV reverse transcriptase. In one preferred embodiment, the reverse transcriptase enzyme comprising a modified MMLV reverse transcriptase amino acid sequence shares at least 50%, 60%, 70%, 75%, 80%, 85%, 90%, 91%, 92%, 93%, 94%, 95%, 96%, 97%, 98%, or 99%, or more sequence identity with SEQ ID NO: 1. In a particularly preferred embodiment, the reverse transcriptase enzyme comprising a modified MMLV reverse transcriptase amino acid sequence shares at least 95%, 96%, 97%, 98%, or 99%, or more sequence identity with SEQ ID NO: 1.

In another embodiment, the reverse transcriptase enzyme comprising a modified MMLV reverse transcriptase amino acid sequence is substantially identical or substantially homologous to a sequence comprising amino acids 1-671 of SEQ ID NOs: 2, 3, or 5. In another embodiment, the reverse transcriptase enzyme comprising a modified MMLV reverse transcriptase amino acid sequence is substantially identical or substantially homologous to a sequence comprising amino acids 1-671 of SEQ ID NOs: 2. In even another embodiment, the reverse transcriptase enzyme comprising a modified MMLV reverse transcriptase amino acid sequence is substantially identical or substantially homologous to a sequence comprising amino acids 1-671 of SEQ ID NOs: 3. In one embodiment, the reverse transcriptase enzyme comprising a modified MMLV reverse transcriptase amino acid sequence is substantially identical or substantially homologous to a sequence comprising amino acids 1-671 of SEQ ID NOs: 5. In one preferred embodiment, the reverse transcriptase enzyme comprising a modified MMLV reverse transcriptase amino acid sequence shares at least 50%, 60%, 70%, 75%, 80%, 85%, 90%, 91%, 92%, 93%, 94%, 95%, 96%, 97%, 98%, or 99%, or more sequence identity with a sequence comprising amino acids 1-671 of SEQ ID NOs: 2, 3, or 5. In a particularly preferred embodiment, the reverse transcriptase enzyme comprising a modified MMLV reverse transcriptase amino acid sequence shares at least 95%, 96%, 97%, 98%, or 99%, or more sequence identity with a sequence comprising amino acids 1-671 of SEQ ID NOs: 2, 3, or 5.

In another embodiment, the reverse transcriptase enzyme comprising a modified MMLV reverse transcriptase amino acid sequence shares at least 50%, 60%, 70%, 75%, 80%, 85%, 90%, 91%, 92%, 93%, 94%, 95%, 96%, 97%, 98%, or 99%, or more sequence identity with a sequence comprising amino acids 1-671 of SEQ ID NO: 1. In a particularly preferred embodiment, the reverse transcriptase enzyme comprising a modified MMLV reverse transcriptase amino acid sequence shares at least 95%, 96%, 97%, 98%, or 99%, or more sequence identity with a sequence comprising amino acids 1-671 of SEQ ID NO: 1.

In yet another embodiment, the mutation at position I416 is any one of I416R, I416H, I416K, I416D, I416E, I416S, I416T, I416N, I416Q, I416C, I416G, I416P, I416A, I416V, I416I, I416L, I416M, I416F, I416Y, I416W. In a preferred embodiment, the mutation at position I416 is any one of I416Q, I416N, I416T, or I416S. In a particularly preferred embodiment, the mutation at position I416 is I416T.

In another embodiment, the mutation at position K267 is any one of K267R, K267H, K267K, K267D, K267E, K267S, K267T, K267N, K267Q, K267C, K267G, K267P, K267A, K267V, K2671, K267L, K267M, K267F, K267Y, K267W. In a preferred embodiment, the mutation at position K267 is any one of K267S, K267N, or K267Q. In another preferred embodiment, the mutation at position K267 is not K267T. In an even more preferred embodiment, the mutation at position K267 is K267N or K267Q. In a particularly preferred embodiment, the mutation at position K267 is K267Q.

In one embodiment, the mutation at position is any one of N674 is N674R, N674H, N674K, N674D, N674E, N674S, N674T, N674N, N674Q, N674C, N674G, N674P, N674A, N674V, N674I, N674L, N674M, N674F, N674Y, N674W. In a preferred embodiment, the mutation at position N674 is any one of N674A, N674V, N674I, N674L, N674M, N674F, N674Y, N674W. In a particularly preferred embodiment, the mutation at position N674 is N674D or N674E. In another particularly preferred embodiment, the mutation at position N674 is N674D.

In one embodiment, the mutation at position is any one of T186 is T186R, T186H, T186K, T186D, T186E, T186S, T186T, T186N, T186Q, T186C, T186G, T186P, T186A, T186V, T186I, T186L, T186M, T186F, T186Y, T186W. In a preferred embodiment, the mutation at position T186 is any one of T186A, T186V, T186I, T186L, T186M, T186F, T186Y, T186W. In another preferred embodiment, the mutation at position T186 is not T186C or T186D. In an even more preferred embodiment, the mutation is selected from the group consisting of T186A, T186V, T186I, T186L, T186M. In another preferred embodiment, the mutation at position T186 is T186A.

In one embodiment, the reverse transcriptase enzyme comprising a modified MMLV reverse transcriptase amino acid sequence has at least two mutations at positions selected from the group consisting of: I416, K267, N674 and T186. In a preferred embodiment, the at least two mutations are at positions I416 and K267. In another preferred embodiment, the at least two mutations are at positions N674 and K267.

In one embodiment, the reverse transcriptase enzyme comprising a modified MMLV reverse transcriptase amino acid sequence comprises at least one further mutation at one or more of the following amino acid positions: P51, H204, N249, T306, F309, D524, E562, K571, D583, T330, N479, L603, M289, M66, L139, D200, T287, H594, E607, Q221, I49, A502, D653, K658, P130, Q237, A307, Y344, Q430, D449, A644, N649, L671, E673, M39, Q91, W388, I179, L333, R390, Q374, E5, E69, E302, W313, L435, N454, D124, E286. In a preferred embodiment, the reverse transcriptase enzyme comprises at least one of the following amino acid mutations: P51L, H204R, N249D, T306K, T306R, T306L, F309N, F309R, D524G, D524A, E562Q, K571R, D583N, T330P, N479D, L603W, L603M, M289V, M66L, L139P, D200N, D200A, D200G, T287A, H594R, H594K, H594Q, E607K, E607G, E607A, Q221R, I49V, I49T, A502V, D653G, D653A, D653H, D653V, K658R, K658Q, P130S, Q237R, A307V, Y344H, Q430R, D449G, D449A, A644V, A644T, N649S, L671P, E673G, E673K, M39V, M39L, Q91R, Q91L, W388R, I179T, I179V, L333Q, R390W, Q374R, E5K, E69K, E302R, E302K, W313F, L435G, L435R, N454K, D124R, E286R.

In another embodiment, the reverse transcriptase enzyme comprising a modified MMLV reverse transcriptase amino acid sequence comprises an I416T mutation, a K267Q mutation, a N674D mutation, and/or a T186A mutation. In another preferred embodiment, the reverse transcriptase enzyme comprising a modified MMLV reverse transcriptase amino acid sequence comprises an I416T mutation, a K267Q mutation, and/or a N674D mutation. In yet another preferred embodiment, the reverse transcriptase enzyme comprising a modified MMLV reverse transcriptase amino acid sequence comprises an I416T mutation and a K267Q mutation.

In another embodiment, the reverse transcriptase enzyme has at least one of the following sets of mutations 1) to 61):
1) I416;
2) K267;
3) N674;
4) T186;
5) I416, K267;
6) K267, N674;
7) I416, M289;
8) I416, H204, T306;
9) I416, D524, D583;
10) I416, H204, T306, F309;
11) I416, H204, T306, F309, M289;
12) I416, H204, T306, F309, D524, D583, M289;
13) I416, H204, T306, F309, D524, E562, D583, M289;
14) I416, P51, H204, N249, D524, E562, K571, D583;
15) I416, P51, H204, N249, D524, E562, K571, D583, M289;
16) I416, P51, H204, N249, T306, F309, D524, E562, K571, D583;
17) I416, P51, H204, N249, T306, F309, D524, E562, K571, D583, M289;
18) K267, M289;
19) K267, H204, T306;
20) K267, D524, D583;
21) K267, H204, T306, F309;
22) K267, H204, T306, F309, M289;
23) K267, H204, T306, F309, D524, D583, M289;
24) K267, H204, T306, F309, D524, E562, D583, M289;
25) K267, P51, H204, N249, D524, E562, K571, D583;
26) K267, P51, H204, N249, D524, E562, K571, D583, M289;
27) K267, P51, H204, N249, T306, F309, D524, E562, K571, D583;
28) K267, P51, H204, N249, T306, F309, D524, E562, K571, D583, M289;
29) I416, K267, M289;
30) I416, K267, H204, T306;
31) I416, K267, D524, D583;
32) I416, K267, H204, T306, F309;
33) I416, K267, H204, T306, F309, M289;
34) I416, K267, H204, T306, F309, D524, D583, M289;
35) I416, K267, H204, T306, F309, D524, E562, D583, M289;
36) I416, K267, P51, H204, N249, D524, E562, K571, D583;
37) I416, K267, P51, H204, N249, D524, E562, K571, D583, M289;
38) I416, K267, P51, H204, N249, T306, F309, D524, E562, K571, D583;
39) I416, K267, P51, H204, N249, T306, F309, D524, E562, K571, D583, M289;
40) N674, M289;
41) N674, H204, T306;
42) N674, D524, D583;
43) N674, H204, T306, F309;
44) N674, H204, T306, F309, M289;
45) N674, H204, T306, F309, D524, D583, M289;
46) N674, H204, T306, F309, D524, E562, D583, M289;
47) N674, P51, H204, N249, D524, E562, K571, D583;
48) N674, P51, H204, N249, D524, E562, K571, D583, M289;
49) N674, P51, H204, N249, T306, F309, D524, E562, K571, D583;
50) N674, P51, H204, N249, T306, F309, D524, E562, K571, D583, M289;
51) T186, M289;
52) T186, H204, T306;
53) T186, D524, D583;
54) T186, H204, T306, F309;
55) T186, H204, T306, F309, M289;
56) T186, H204, T306, F309, D524, D583, M289;
57) T186, H204, T306, F309, D524, E562, D583, M289;
58) T186, P51, H204, N249, D524, E562, K571, D583;
59) T186, P51, H204, N249, D524, E562, K571, D583, M289;
60) T186, P51, H204, N249, T306, F309, D524, E562, K571, D583;
61) T186, P51, H204, N249, T306, F309, D524, E562, K571, D583, M289.

In another embodiment, the reverse transcriptase enzyme has at least one of the following sets of mutations 62) to 186):
62) I416, T330;
63) I416, N479;
64) I416, L603;
65) I416, M66;
66) I416, D200;
67) I416, T287;
68) I416, L139;
69) I416, L139, H594;
70) I416, L139, E607;
71) I416, L139, D653;
72) I416, T330, N479, L139;
73) I416, T330, L603, L139;
74) I416, N479, L603, L139;
75) I416, L603, L139;
76) I416, T330, L139;
77) I416, D200, L139, E607;
78) I416, T330, L139, D653;
79) I416, T330, N479, L603, L139;
80) I416, T330, L603, D200, L139;
81) I416, L603, D200, L139, D653;
82) I416, D200, L139, E607;
83) I416, T330, L603, D200, L139, D653;
84) I416, N479, L603, D200, L139, H594;
85) I416, T330, L603, D200, L139, E607;
86) I416, T330, L603, D200, L139, E607;
87) K267, T330, L139;
88) K267, N479, L139;
89) K267, L603, L139;
90) K267, M66, L139;
91) K267, D200, L139;
92) K267, T287, L139;
93) K267, L139;
94) K267, L139, H594;
95) K267, L139, E607;
96) K267, L139, D653;
97) K267, T330, N479, L139;
98) K267, T330, L603, L139;
99) K267, N479, L603, L139;
100) K267, L603, L139;
101) K267, T330, L139;
102) K267, D200, L139, E607;
103) K267, T330, L139, D653;
104) K267, T330, N479, L603, L139;
105) K267, T330, L603, D200, L139;
106) K267, L603, D200, L139, D653;
107) K267, D200, L139, E607;
108) K267, T330, L603, D200, L139, D653;
109) K267, N479, L603, D200, L139, H594;
110) K267, T330, L603, D200, L139, E607;
111) K267, T330, L603, D200, L139, E607;
112) I416, K267, T330, L139;
113) I416, K267, N479, L139;
114) I416, K267, L603, L139;
115) I416, K267, M66, L139;
116) I416, K267, D200, L139;
117) I416, K267, T287, L139;
118) I416, K267, L139;
119) I416, K267, L139, H594;
120) I416, K267, L139, E607;
121) I416, K267, L139, D653;
122) I416, K267, T330, N479, L139;
123) I416, K267, T330, L603, L139;
124) I416, K267, N479, L603, L139;
125) I416, K267, L603, L139;
126) I416, K267, T330, L139;
127) I416, K267, D200, L139, E607;
128) I416, K267, T330, L139, D653;
129) I416, K267, T330, N479, L603, L139;
130) I416, K267, T330, L603, D200, L139;
131) I416, K267, L603, D200, L139, D653;
132) I416, K267, D200, L139, E607;
133) I416, K267, T330, L603, D200, L139, D653;
134) I416, K267, N479, L603, D200, L139, H594;
135) I416, K267, T330, L603, D200, L139, E607;
136) I416, K267, T330, L603, D200, L139, E607;
137) N674, T330, L139;
138) N674, N479, L139;
139) N674, L603, L139;
140) N674, M66, L139;
141) N674, D200, L139;
142) N674, T287, L139;
143) N674, L139;
144) N674, L139, H594;
145) N674, L139, E607;
146) N674, L139, D653;
147) N674, T330, N479, L139;
148) N674, T330, L603, L139;
149) N674, N479, L603, L139;
150) N674, L603, L139;
151) N674, T330, L139;
152) N674, D200, L139, E607;
153) N674, T330, L139, D653;
154) N674, T330, N479, L603, L139;
155) N674, T330, L603, D200, L139;
156) N674, L603, D200, L139, D653;
157) N674, D200, L139, E607;
158) N674, T330, L603, D200, L139, D653;
159) N674, N479, L603, D200, L139, H594;
160) N674, T330, L603, D200, L139, E607;
161) N674, T330, L603, D200, L139, E607;
162) T186, T330, L139;
163) T186, N479, L139;
164) T186, L603, L139;
165) T186, M66, L139;
166) T186, D200, L139;
167) T186, T287, L139;
168) T186, L139;
169) T186, L139, H594;
170) T186, L139, E607;
171) T186, L139, D653;
172) T186, T330, N479, L139;
173) T186, T330, L603, L139;
174) T186, N479, L603, L139;
175) T186, L603, L139;
176) T186, T330, L139;
177) T186, D200, L139, E607;
178) T186, T330, L139, D653;
179) T186, T330, N479, L603, L139;
180) T186, T330, L603, D200, L139;
181) T186, L603, D200, L139, D653;
182) T186, D200, L139, E607;
183) T186, T330, L603, D200, L139, D653;
184) T186, N479, L603, D200, L139, H594;
185) T186, T330, L603, D200, L139, E607;
186) T186, T330, L603, D200, L139, E607.

In another embodiment, the reverse transcriptase enzyme has at least one of the following sets of mutations 187) to 256):
187) I416, E69;
188) I416, E302;
189) I416, W313;
190) I416, L435;
191) I416, N454;
192) I416, D124;
193) I416, E286;
194) I416, E69, E302;
195) I416, E302, W313;
196) I416, E302, W313, L435;
197) I416, E69, E302, W313, L435;
198) I416, E302, W313, L435, N454;
199) I416, E69, E302, W313, L435, N454;
200) I416, E69, E302, W313, L435, N454, D124, E286;
201) K267, E69;
202) K267, E302;
203) K267, W313;
204) K267, L435;
205) K267, N454;
206) K267, D124;
207) K267, E286;
208) K267, E69, E302;
209) K267, E302, W313;
210) K267, E302, W313, L435;
211) K267, E69, E302, W313, L435;
212) K267, E302, W313, L435, N454;
213) K267, E69, E302, W313, L435, N454;
214) K267, E69, E302, W313, L435, N454, D124, E286;
215) I416, K267, E69;
216) I416, K267, E302;
217) I416, K267, W313;
218) I416, K267, L435;
219) I416, K267, N454;
220) I416, K267, D124;
221) I416, K267, E286;
222) I416, K267, E69, E302;
223) I416, K267, E302, W313;
224) I416, K267, E302, W313, L435;
225) I416, K267, E69, E302, W313, L435;
226) I416, K267, E302, W313, L435, N454;
227) I416, K267, E69, E302, W313, L435, N454;
228) I416, K267, E69, E302, W313, L435, N454, D124, E286;
229) N674, E69;
230) N674, E302;
231) N674, W313;
232) N674, L435;
233) N674, N454;
234) N674, D124;
235) N674, E286;
236) N674, E69, E302;
237) N674, E302, W313;
238) N674, E302, W313, L435;
239) N674, E69, E302, W313, L435;
240) N674, E302, W313, L435, N454;
241) N674, E69, E302, W313, L435, N454;
242) N674, E69, E302, W313, L435, N454, D124, E286;
243) T186, E69;
244) T186, E302;
245) T186, W313;
246) T186, L435;
247) T186, N454;
248) T186, D124;
249) T186, E286;
250) T186, E69, E302;
251) T186, E302, W313;
252) T186, E302, W313, L435;
253) T186, E69, E302, W313, L435;
254) T186, E302, W313, L435, N454;
255) T186, E69, E302, W313, L435, N454;
256) T186, E69, E302, W313, L435, N454, D124, E286;

In another embodiment, the reverse transcriptase enzyme has at least one of the following sets of mutations 257) to 311):

257) 7 and one of 62-186; 258) 8 and one of 62-186; 259) 9 and one of 62-186; 260) 10 and one of 62-186; 261) 11 and one of 62-186; 262) 12 and one of 62-186; 263) 13 and one of 62-186; 264) 14 and one of 62-186; 265) 15 and one of 62-186; 266) 16 and one of 62-186; 267) 17 and one of 62-186; 268) 18 and one of 62-186; 269) 19 and one of 62-186; 270) 20 and one of 62-186; 271) 21 and one of 62-186; 272) 22 and one of 62-186; 273) 23 and one of 62-186; 274) 24 and one of 62-186; 275) 25 and one of 62-186; 276) 26 and one of 62-186; 277) 27 and one of 62-186; 278) 28 and one of 62-186; 279) 29 and one of 62-186; 280) 30 and one of 62-186; 281) 31 and one of 62-186; 282) 32 and one of 62-186; 283) 33 and one of 62-186; 284) 34 and one of 62-186; 285) 35 and one of 62-186; 286) 36 and one of 62-186; 287) 37 and one of 62-186; 288) 38 and one of 62-186; 289) 39 and one of 62-186; 290) 40 and one of 62-186; 291) 41 and one of 62-186; 292) 42 and one of 62-186; 293) 43 and one of 62-186; 294) 44 and one of 62-186; 295) 45 and one of 62-186; 296) 46 and one of 62-186; 297) 47 and one of 62-186; 298) 48 and one of 62-186; 299) 49 and one of 62-186; 300) 50 and one of 62-186; 301) 51 and one of 62-186; 302) 52 and one of 62-186; 303) 53 and one of 62-186; 304) 54 and one of 62-186; 305) 55 and one of 62-186; 306) 56 and one of 62-186; 307) 57 and one of 62-186; 308) 58 and one of 62-186; 309) 59 and one of 62-186; 310) 60 and one of 62-186; 311) 61 and one of 62-186.

In another embodiment, the reverse transcriptase enzyme has at least one of the following sets of mutations 312) to 366):

312) 7 and one of 187-256; 313) 8 and one of 187-256; 314) 9 and one of 187-256; 315) 10 and one of 187-256; 316) 11 and one of 187-256; 317) 12 and one of 187-256; 318) 13 and one of 187-256; 319) 14 and one of 187-256; 320) 15 and one of 187-256; 321) 16 and one of 187-256; 322) 17 and one of 187-256; 323) 18 and one of 187-256; 324) 19 and one of 187-256; 325) 20 and one of 187-256; 326) 21 and one of 187-256; 327) 22 and one of 187-256; 328) 23 and one of 187-256; 329) 24 and one of 187-256; 330) 25 and one of 187-256; 331) 26 and one of 187-256; 332) 27 and one of 187-256; 333) 28 and one of 187-256; 334) 29 and one of 187-256; 335) 30 and one of 187-256; 336) 31 and one of 187-256; 337) 32 and one of 187-256; 338) 33 and one of 187-256; 339) 34 and one of 187-256; 340) 35 and one of 187-256; 341) 36 and one of 187-256; 342) 37 and one of 187-256; 343) 38 and one of 187-256; 344) 39 and one of 187-256; 345) 40 and one of 187-256; 346) 41 and one of 187-256; 347) 42 and one of 187-256; 348) 43 and one of 187-256; 349) 44 and one of 187-256; 350) 45 and one of 187-256; 351) 46 and one of 187-256; 352) 47 and one of 187-256; 353) 48 and one of 187-256; 354) 49 and one of 187-256; 355) 50 and one of 187-256; 356) 51 and one of 187-256; 357) 52 and one of 187-256; 358) 53 and one of 187-256; 359) 54 and one of 187-256; 360) 55 and one of 187-256; 361) 56 and one of 187-256; 362) 57 and one of 187-256; 363) 58 and one of 187-256; 364) 59 and one of 187-256; 365) 60 and one of 187-256; 366) 61 and one of 187-256.

In another embodiment, the reverse transcriptase enzyme has at least one of the following sets of mutations 367) to 491):

367) 62 and one of 187-256; 368) 63 and one of 187-256; 369) 64 and one of 187-256; 370) 65 and one of 187-256; 371) 66 and one of 187-256; 372) 67 and one of 187-256; 373) 68 and one of 187-256; 374) 69 and one of 187-256; 375) 70 and one of 187-256; 376) 71 and one of 187-256; 377) 72 and one of 187-256; 378) 73 and one of 187-256; 379) 74 and one of 187-256; 380) 75 and one of 187-256; 381) 76 and one of 187-256; 382) 77 and one of 187-256; 383) 78 and one of 187-256; 384) 79 and one of 187-256; 385) 80 and one of 187-256; 386) 81 and one of 187-256; 387) 82 and one of 187-256; 388) 83 and one of 187-256; 389) 84 and one of 187-256; 390) 85 and one of 187-256; 391) 86 and one of 187-256; 392) 87 and one of 187-256; 393) 88 and one of 187-256; 394) 89 and one of 187-256; 395) 90 and one of 187-256; 396) 91 and one of 187-256; 397) 92 and one of 187-256; 398) 93 and one of 187-256; 399) 94 and one of 187-256; 400) 95 and one of 187-256; 401) 96 and one of 187-256; 402) 97 and one of 187-256; 403) 98 and one of 187-256; 404) 99 and one of 187-256; 405) 100 and one of 187-256; 406) 101 and one of 187-256; 407) 102 and one of 187-256; 408) 103 and one of 187-256; 409) 104 and one of 187-256; 410) 105 and one of 187-256; 411) 106 and one of 187-256; 412) 107 and one of 187-256; 413) 108 and one of 187-256; 414) 109 and one of 187-256; 415) 110 and one of 187-256; 416) 111 and one of 187-256; 417) 112 and one of 187-256; 418) 113 and one of 187-256; 419) 114 and one of 187-256; 420) 115 and one of 187-256; 421) 116 and one of 187-256; 422) 117 and one of 187-256; 423) 118 and one of 187-256; 424) 119 and one of 187-256; 425) 120 and one of 187-256; 426) 121 and one of 187-256; 427) 122 and one of 187-256; 428) 123 and one of 187-256; 429) 124 and one of 187-256; 430) 125 and one of 187-256; 431) 126 and one of 187-256; 432) 127 and one of 187-256; 433) 128 and one of 187-256; 434) 129 and one of 187-256; 435) 130 and one of 187-256; 436) 131 and one of 187-256; 437) 132 and one of 187-256; 438) 133 and one of 187-256; 439) 134 and one of 187-256; 440) 135 and one of 187-256; 441) 136 and one of 187-256; 442) 137 and one of 187-256; 443) 138 and one of 187-256; 444) 139 and one of 187-256; 445) 140 and one of 187-256; 446) 141 and one of 187-256; 447) 142 and one of 187-256; 448) 143 and one of 187-256; 449) 144 and one of 187-256; 450) 145 and one of 187-256; 451) 146 and one of 187-256; 452) 147 and one of 187-256; 453) 148 and one of 187-256; 454) 149 and one of 187-256; 455) 150 and one of 187-256; 456) 151 and one of 187-256; 457) 152 and one of 187-256; 458) 153 and one of 187-256; 459) 154 and one of 187-256; 460) 155 and one of 187-256; 461) 156 and one of 187-256; 462) 157 and one of 187-256; 463) 158 and one of 187-256; 464) 159 and one of 187-256; 465) 160 and one of 187-256; 466) 161 and one of 187-256; 467) 162 and one of 187-256; 468) 163 and one of 187-256; 469) 164 and one of 187-256; 470) 165 and one of 187-256; 471) 166 and one of 187-256; 472) 167 and one of 187-256; 473) 168 and one of 187-256; 474) 169 and one of 187-256; 475) 170 and one of 187-256; 476) 171 and one of 187-256; 477) 172 and one of 187-256; 478) 173 and one of 187-256; 479) 174 and one of 187-256; 480) 175 and one of 187-256; 481) 176 and one of 187-256; 482) 177 and one of 187-256; 483) 178 and one of 187-256; 484) 179 and one of 187-256; 485) 180 and one of 187-256; 486) 181 and one of 187-256; 487) 182 and one of 187-256; 488) 183 and one of 187-256; 489) 184 and one of 187-256; 490) 185 and one of 187-256; 491) 186 and one of 187-256.

In preferred embodiments, the reverse transcriptase has at least one of the sets of mutations 1) to 491, wherein said reverse transcriptase comprises at least one of the following amino acid mutations: P51L, H204R, N249D, T306K, T306R, T306L, F309N, F309R, D524G, D524A, E562Q, K571R, D583N, T330P, N479D, L603W, L603M, M289V, M66L, L139P, D200N, D200A, D200G, T287A, H594R, H594K, H594Q, E607K, E607G, E607A, D653G, D653A, D653H, D653V, K658R, K658Q, E69K, E302R, E302K, W313F, L435G, L435R, N454K, D124R, E286R.

In another embodiment, the reverse transcriptase enzyme has at least one of the sets of mutations 1) to 491), wherein said reverse transcriptase enzyme further comprises at least one mutation at an amino acid position selected from Q221, I49, A502, K658, P130, Q237, A307, Y344, Q430, D449, A644, N649, L671, E673, M39, Q91, W388, I179, L333, R390, Q374, E5. In preferred embodiments, said reverse transcriptase comprises at least one of the following amino acid mutations: Q221R, I49V, I49T, A502V, P130S, Q237R, A307V, Y344H, Q430R, D449G, D449A, A644V, A644T, N649S, L671P, E673G, E673K, M39V, M39L, Q91R, Q91L, W388R, I179T, I179V, L333Q, R390W, Q374R, E5K.

### Inhibitor Resistance

In some embodiments, the reverse transcriptase enzyme confers increased resistance to inhibitors. In one embodiment, said inhibitors are PCR inhibitors such as antiviral substances, haemoglobin, IgG, myoglobin, hemin lactoferrin, hormones, heparin, (complex) polysaccharides, lipids, urate, bile salts, glycogen, (poly)phenols, pectin, xylan, proteases, metal ions, calcium ions, debris, fulvic acids, porphyrins, polyphenols, formalin, paraffin, humic material, bone dust, coprolite, peat extract, clay-rich soil, denaturants. In a preferred embodiment, the reverse transcriptase enzyme confers increased resistance to porphyrins, polyphenols, humic material and denaturants. In a particularly preferred embodiment, the reverse transcriptase enzyme confers increased resistance to hemin, tannic acid, humic acid, and guanidine isothiocyanate. In another particularly preferred embodiment, the reverse transcriptase according to the invention confers increased resistance to heparin, xylan, isopropanol and ELUGENT™ detergent. In one embodiment, the reverse transcriptase enzyme demonstrates increased resistance compared to wild-type MMLV reverse transcriptase, such that the reverse transcriptase according to the invention is capable of reverse transcription in the presence of up to 15 μM hemin, up to 7.5 ng/μL tannic acid, up to 10 ng/μL humic acid and up to 80 mM guanidine isothiocyanate.

### Further Improved Properties

In one embodiment, the reverse transcriptase enzyme according to the invention leads to increased cDNA synthesis speed by at least 1%, 5%, 10%, 15%, 20%, 25%, 30%, 35%, 40%, 45%, 50%, 55%, 60%, 65%, 70%, 75%, 80%, 85%, 90%, 95% compared to wild-type MMLV reverse transcriptase, as determined by nucleotide incorporation via reverse transcriptase using deoxythymidine triphosphate (dTTP) labelled with ³H on the methyl group (also known as tritiated dTTP). Usually, the nucleotide incorporation (here: monitoring cDNA synthesis speed) is determined in units/mg, observed over a pre-defined time interval. The skilled person knows how to conduct said determination.

In another embodiment, the reverse transcriptase enzyme according to the invention leads to increased cDNA synthesis speed, the cDNA synthesis speed being measured by the C_{q} value and compared to the C_{q} value of wild-type MMLV reverse transcriptase. Hence, in yet another embodiment, the reverse transcriptase enzyme according to the invention possesses increased inhibitor resistance and leads to increased cDNA synthesis speed compared to wild-type MMLV reverse transcriptase.

In another embodiment, the reverse transcriptase enzyme according to the invention leads to increased processivity compared to wild-type MMLV reverse transcriptase. In a particularly preferred embodiment, the reverse transcriptase enzyme according to the invention can add 100, 200, 300, 400, 500, 600, 700, 800, 900, 1000, 1100, 1200, 1300, 1400, 1500, 1600, 1700, 1800, 1900 or 2000 nucleotides in a single binding event. In another embodiment, the reverse transcriptase enzyme according to the invention can add 500, 1000 or 1500 nucleotides in a single binding event, corresponding to a processivity that is about 22, 43, 65 greater than the processivity of wild-type MMLV reverse transcriptase.

In yet another embodiment, the reverse transcriptase enzyme according to the invention has a higher affinity for the template of interest compared to wild-type MMLV reverse transcriptase. The affinity for the template of interest is typically measured by the equilibrium dissociation constant (K_{D}). In a preferred embodiment, the K_{D} Of the reverse transcriptase enzyme according to the invention is smaller than the K_{D}Of wild-type MMLV reverse transcriptase.

In yet another embodiment, the reverse transcriptase enzyme has an optimum activity at a temperature of 50° C., 51° C., 52° C., 53° C., 54° C., 55° C., 56° C., 57° C., 58° C., 59° C., 60° C. In even another embodiment, the reverse transcriptase enzyme has a higher activity at a temperature of 50° C., 51° C., 52° C., 53° C., 54° C., 55° C., 56° C., 57° C., 58° C., 59° C., 60° C. compared to wild-type MMLV reverse transcriptase. In one embodiment, the reverse transcriptase enzyme has a higher activity at a temperature of 55° C. compared to wild-type MMLV reverse transcriptase. In a preferred embodiment, the reverse transcriptase enzyme having a higher activity at a temperature of 55° C. compared to wild-type MMLV reverse transcriptase is a reverse transcriptase enzyme comprising an I416T mutation, a K267Q mutation, a N674D mutation, or a T186A mutation. In a particularly preferred embodiment, the reverse transcriptase enzyme having a higher activity at a temperature of 55° C. compared to wild-type MMLV reverse transcriptase is a reverse transcriptase enzyme comprising an I416T mutation. In another embodiment, the thermal stability is determined by measuring the activity of the reverse transcriptase enzyme after thermal treatment. In one embodiment, the thermal activity is increased by at least 10%, 20%, 30%, 40%, 50% compared to wild-type MMLV reverse transcriptase.

### Uses of the Reverse Transcriptase

In another aspect of the invention, the reverse transcriptase according to any of the preceding embodiment is used for reverse transcription of RNA. Reverse transcription is used in various laboratory application. Therefore, in one embodiment, the reverse transcriptase according to any of the preceding embodiments is used in laboratory applications such as reverse transcription polymerase chain reaction (RT-PCR), quantitative RT-PCR (RT-qPCR), cDNA cloning and library construction, rapid amplification of cDNA ends (RACE), gene expression microarrays, RNA sequencing (RNA-Seq), reverse transcription loop-mediated isothermal amplification (RT-LAMP), single cell and next-generation sequencing, or dPCR.

Therefore, in a preferred embodiment, the reverse transcriptase of the present invention may be used to synthesise DNA using RNA as a template, thereby obtaining complementary DNA (cDNA) for RT-PCR, or RT-qPCR.

In another preferred embodiment, the RT-qPCR is one-step RT-qPCR or two-step RT-qPCR. In a preferred embodiment, the RT-qPCR is one-step RT-qPCR. In an equally preferred embodiment, the RT-PCR is two-step RT-qPCR.

In yet another embodiment, the RT-PCR is one-step RT-PCR or two-step RT-PCR. In a preferred embodiment, the RT-PCR is one-step RT-PCR. In an equally preferred embodiment, the RT-PCR is two-step RT-PCR.

### Compositions and Reaction Mixtures Comprising Reverse Transcriptases

The present invention also provides compositions and reaction mixtures comprising a variety of components in various combinations. In some embodiments of the present invention, the compositions are formulated by admixing one or more reverse transcriptases. In some other embodiments, the compositions are formulated in a buffered salt solution. One or more DNA polymerases and/or one or more nucleotides, and/or one or more primers may optionally be added to the compositions of the invention. Alternatively, the reaction mixture according to the invention can comprise one or more DNA polymerases and/or one or more nucleotides, and/or one or more primers. These compositions and/or reaction mixtures can be used in the present invention for reverse transcription, followed by one-step PCR or one-step qPCR. Alternatively, the compositions and/or reaction mixtures can be used for reverse transcription, followed by two-step PCR or one-step qPCR.

In some embodiments, the reverse transcriptase according to the invention is provided at working concentrations (e.g., 1×) in stable buffered salt solutions. Such compositions can also be formulated as concentrated stock solutions (e.g., 2×, 3×, 4×, 5×, 6×, 10×, etc.). In some embodiments, having the composition as a concentrated (e.g., 5×) stock solution allows a greater amount of nucleic acid sample to be added (such as, for example, when the compositions are used for nucleic acid synthesis).

In addition to the enzyme components, the compositions according to the invention can comprise one or more buffers and cofactors necessary for synthesis of a nucleic acid molecule such as a cDNA molecule. In some embodiments, buffers for use in forming the present compositions are acetate, sulfate, hydrochloride, phosphate or free acid forms of Tris-(hydroxymethyl)aminomethane (TRIS™) or 4-(2-hydroxyethyl)-1-piperazineethanesulfonic acid (HEPES), although alternative buffers of the same approximate ionic strength and pKa as TRIS™ or HEPES may be used with equivalent results. For example, possible buffers for use with the described enzymes can include 3-{[tris(hydroxymethyl)methyl]amino}propanesulfonic acid (TAPS), N,N-bis(2-hydroxyethyl)glycine (Bicine), Tris-(hydroxymethyl)aminomethane (Tris™), N-tris(hydroxymethyl)methylglycine (Tricine), 3-[N-Tris(hydroxymethyl)methylamino]-2-hydroxypropanesulfonic Acid (TAPSO), 4-2-hydroxyethyl-1-piperazineethanesulfonic acid (HEPES), 2-{[tris(hydroxymethyl)methyl]amino}ethanesulfonic acid (TES), 3-(N-morpholino)propanesulfonic acid (MOPS), piperazine-N,N-bis(2-ethanesulfonic acid) (PIPES), and dimethylarsinic acid (cacodylate).

In addition to buffer salts, cofactor salts such as those of potassium (preferably potassium chloride or potassium acetate) and magnesium (preferably magnesium chloride or magnesium acetate are contemplated for use in the compositions of the invention.

Addition of one or more carbohydrates and/or sugars to the compositions and/or reaction mixtures may also be advantageous, to support enhanced stability of the compositions upon storage and/or reaction mixtures during synthesis. In some embodiments, carbohydrates or sugars for inclusion in the compositions and/or reaction mixtures of the invention include sucrose, trehalose, glycerol, and the like. In some embodiments, trehalose is provided at concentrations ranging from 0.01M to 5M (e.g., 0.01 M, 0.05 M, 0.1 M, 0.5 M, 0.75 M, 1.0 M, 2.0 M, 3.0 M, 4.0 M or 5.0 M). In some embodiments, glycerol is provided at concentrations ranging from 5% to 60%. (e.g., 5%, 10%, 15%, 25%, 30%, 40%, 50%, 60%).

Likewise, addition of one or more surfactants and/or detergents to the compositions and/or reaction mixtures may also be advantageous, to support enhanced stability of the compositions and/or reaction mixtures upon storage. Preferred detergents for inclusion in the compositions and/or reaction mixtures of the invention include Tween™20, Nonidet™ P 40 (NP-40), Brij™58, Triton™ X-100, Ecosurf™ SA, Ecosurf™ EH-9, Tergitol™, CHAPS, and the like. In some embodiments, the addition of one or more of detergents Tween™20, Nonidet™ P40 (NP-40), Brij™58, Triton™ X-100, Ecosurf™ SA-9, Ecosurf™ EH-9, Tergitol™, CHAPS, may enhance enzyme activity in the reaction mixtures.

In some embodiments, component deterioration can be reduced by storage of the compositions and/or reaction mixtures at a temperature of about −80° C. (for up to two years) or at a temperature of about −20° C. (for up to one year).

In some embodiments, the compositions and/or reaction mixtures of the invention can be packaged in a suitable container or vessel capable of holding the composition and which will not significantly interact with components of the composition. The container or vessel can be designed to permit easy dispensing of the dosage form by individuals or by a liquid handling instrument. The containers or vessels of such composition and/or reaction mixture can be further packaged into multi-pack units.

In another aspect, the compositions and reverse transcriptases of the invention may be prepared and stored in dry form (e.g., lyophilised) in the presence of one or more carbohydrates, sugars, or synthetic polymers. Preferred carbohydrates, sugars or polymers for the preparation of dried compositions or reverse transcriptases include, sucrose, trehalose, and polyvinylpyrrolidone (PVP) or combinations thereof (see e.g., U.S. Pat. Nos. 5,098,893, 4,891,319, and 5,556,771). Such dried compositions and enzymes may be stored at various temperatures for extended times without significant deterioration of enzymes or components of the compositions of the invention. In some preferred embodiments, the dried reverse transcriptases or compositions are stored at about −20° C. to about 25° C.

In another aspect, the compositions and reverse transcriptases of the invention may be prepared as stabilized glycerol-free enzyme formulations. Such formulations may have the advantage of being lyophilizable in comparison to formulations with glycerol. In some embodiments, a glycerol-free solution has no glycerol. In some embodiments, a glycerol-free solution has nominal glycerol or glycerol that is undetectable. In some embodiments, a glycerol-free buffer comprises no more than 2% glycerol, preferably, no more than 1% glycerol, more preferably, no more than 0.5% glycerol, or, even more preferably, no more than 0.1% glycerol. In some embodiments, the stabilized enzyme formulation comprises stabilizing concentrations of K+ and/or Na+ based salts (see, e.g. U.S. Pat. No. 11,268,084). In some embodiments, the stabilized enzyme further comprises at least one of a buffer salt(s); reducing agent(s); detergent(s); cryoprotectant(s); and/or optional other stabilizer(s).

The invention further includes compositions and/or reaction mixtures for reverse transcribing nucleic acid molecules, as well as reverse transcription methods employing such compositions and/or reaction mixtures and nucleic acid molecules produced using such methods. In some embodiments, compositions of the invention may contain one or more of the following components: (1) one or more buffering agent (e.g., sodium phosphate, sodium acetate, 2-(N-morpholino)-ethanesulfonic acid (MES), tris-(hydroxymethyl)aminomethane (Tris), 3-(cyclohexylamino)-2-hydroxy-1-propanesulfonic acid (CAPS), citrate, N-2-hydroxyethylpiperazine-N-2-ethanesulfonic acid (HEPES), acetate, 3-(N-morpholino)propanesulfonic acid (MOPS), N-tris(hydroxymethyl)methyl-3-aminopropanesulfonio acid (TAPS), etc.), (2) one or more monovalent cationic salt (e.g., NaCl, KCl, etc.), (3) one or more divalent cationic salt (e.g., MnCl2, MgCl₂, MgSO₄, CaCl₂, etc.), (4) one or more reducing agent (e.g., dithiothreitol, β-mercaptoethanol, etc.), (5) one or more ionic or non-ionic detergent (e.g., TRITON™ X-100, NONIDET™ P40 Ecosurf™ SA-9, Ecosurf™ EH-9, Tergitol™, etc.), (6) nucleotides (e.g., dNTPs, such as dGTP, dATP, dCTP, dTTP, etc.), (7) RNA to be reverse transcribed and/or amplified, (8) one or more RNase inhibitor (e.g., RNASEOUT™, Invitrogen Corporation, Carlsbad, Calif., catalog number 10777-019 etc.), (9) a reverse transcriptase e.g., a reverse transcriptase of the invention, and/or (10) one or more diluent (e.g., water). Other components and/or constituents (e.g., primers, DNA polymerases, etc.) may also be present in compositions and/or reaction mixtures.

The concentration of the buffering agent in the compositions and/or reaction mixtures of the invention will vary with the particular buffering agent used. Typically, the working concentration of the buffering agent will be from about 5 mM to about 500 mM.

The final pH of solutions of the invention will generally be set and maintained by buffering agents present in compositions and/or reaction mixtures of the invention. The pH of compositions of the invention, and hence reaction mixtures of the invention, will vary with the particular use and the buffering agent present but will often be from about pH 5.5 to about pH 9.0.

As indicated, one or more monovalent cationic salts (e.g., NaCl, KCl, etc.) may be included in compositions and/or reaction mixtures of the invention. In many instances, salts used in compositions of the invention will dissociate in solution to generate at least one species which is monovalent (e.g., Na⁺, K⁺, etc.) When included in compositions of the invention, salts will often be present either individually or in a combined concentration of from about 0.5 mM to about 500 mM.

As indicated, one or more divalent cationic salts (e.g., MnCl₂, MgCl₂, MgSO₄, CaCl₂), etc.) may be included in compositions and/or reaction mixtures of the invention. In many instances, salts used in compositions and/or reaction mixtures of the invention will dissociate in solution to generate at least one species which is monovalent (e.g., Mg⁺⁺, Mn⁺⁺, Ca⁺⁺, etc.). When included in compositions and/or reaction mixtures of the invention, salts will often be present either individually or in a combined concentration of from about 0.5 mM to about 500 mM.

When included in compositions and/or reaction mixtures of the invention, reducing agents (e.g., dithiothreitol, β-mercaptoethanol, TCEP, etc.) will often be present either individually or in a combined concentration of from about 0.1 mM to about 50 mM.

Other additives capable of facilitating or enhancing reverse transcription, amplification, or a combination of both reactions (e.g., agents for facilitating or enhancing RT-PCR), other than those disclosed herein, are known in the art. In accordance with the present invention, one or more of these additives can be incorporated to optimize the generation and replication of nucleic acids from a ribonucleic acid or deoxyribonucleic acid templates. Additives can be organic or inorganic compounds. Some additives useful in the present invention include polypeptides as well as nonpolypeptide additives. Such additives can include, for example, RNase inhibitor protein (RIP), uracil DNA glycosylase (UDG), lectins, E. coli single-stranded binding (SSB) protein, tRNA, rRNA, 7-deaza-2-deoxyguanosine (dC7GTP) sulfur-containing compounds, acetate-containing compounds, dimethylsulfoxide (DMSO), ribonuclease inhibitor (e.g., Rnase OUT™) formamide, betaine, tetramethylammonium chloride (TMAC), polyethylene glycol (PEG), ectoine, sodium azide, kathon, and polyols, to name just a few. Those of ordinary skill in the art will be able to identify additional additives for use in accordance with the present invention.

Compositions and/or reaction mixtures of the invention may also comprise one or more hot start components. Hot-start is a common technique used to reduce nonspecific amplification due to assembly of nucleic acid synthesis reactions at room temperature. In some embodiments, the reverse transcriptase is reversibly inactivated or physically separated from one or more critical components in the reaction. For example, magnesium can be sequestered in a wax bead, which melts as the reaction is heated, releasing the component only at higher temperatures (see, e.g., Carothers et al. 1989; Krishnan et al. 1991; Clark, 1988). The reverse transcriptase can also be kept in an inactive state by binding to an oligonucleotide, also known as an aptamer (see, e.g., Lin and Jayasena, 1997; Dang and Jayasena, 1996) or an antibody (see, e.g., Scalice et al. 1994; Sharkey et al, 1994). This bond can then be disrupted at a higher temperature, releasing the functional reverse transcriptase.

In yet other embodiments, the reverse transcriptase can be maintained in an inactive state through chemical modification (see, e.g., Moretti, T. et al 1998). In some embodiments, the chemical modification is reversible. Thus, in some embodiments, the reverse transcriptase is chemically modified such that it is in an inactive state at a lower temperature (e.g., less than about 55° C.) and is fully functional/active at an elevated temperature (e.g., greater than about 55° C.).

In some embodiments, nucleotides e.g., dNTPs, such as dGTP, dATP, dCTP, dTTP, etc. will be present in reaction mixtures of the invention.

In some embodiments, the composition and/or reaction mixture comprises at least one protein stabilizer. In some embodiments, the protein stabilizer is selected from BSA, inactive polymerase, and apotransferrin.

In some embodiments, the reaction mixture comprises at least one dye or fluorophore. Dyes that may be used in the compositions provided herein include xylene cyanol FF, tartrazine, phenol red, quinoline yellow, Brilliant Blue, Patent Blue, indigocarmine, acid red 1, m-cresol purple, cresol red, neutral red, bromocresol green, acid violet 5, bromo phenol blue, orange G (see, e.g., US20190270975A1, U.S. Pat. No. 8,663,925 B2). Additional exemplary dyes are described, e.g., in U.S. Pat. No. 6,942,964. The skilled person will appreciate that any dye that does not inhibit nucleic acid synthesis by polymerases may be used.

In some embodiments, the composition and/or reaction mixture comprises at least one agent that increases the density of the composition. In some embodiments, the composition and/or reaction mixture comprises at least one agent selected from PEG 4000 and/or sucrose.

Compositions and/or reaction mixtures of the invention may be prepared as concentrated solutions (e.g., 5× solutions) which are diluted to a working concentration for final use.

In some embodiments, compositions and/or reaction mixtures of the invention will be provided in sterile form. Sterilization may be performed on the individual components of compositions and/or reaction mixtures prior to mixing or on compositions after they are prepared. Sterilization of such solutions may be performed by any suitable means including autoclaving or ultrafiltration.

### Kits

In yet another aspect, the invention provides a kit comprising the reverse transcriptase according to any of the preceding embodiments. In one embodiment, the kit may comprise additional components in addition to the reverse transcriptase of the present invention such as further reagents suitable for PCR such as DNA polymerase, or primers, one or more buffers, nuclease-free water, salts such as magnesium sulphate, one or more dyes/fluorophores, ribonucleotides, deoxyribonucleotides etc.

In some embodiments, said additional components may be present in a master mix. In a preferred embodiment, said additional components and the reverse transcriptase of the invention are present in a master mix. Furthermore, the kit may also comprise reaction vessels, controls, and an instruction manual.

In one embodiment, the kit is a two-step RT-qPCR kit. In a preferred embodiment, the kit is a one-step RT-qPCR kit.

### Polynucleotides

In even another aspect, the invention provides a polynucleotide encoding the reverse transcriptase enzyme according to any preceding embodiment.

In some embodiments, the polynucleotide may be DNA or RNA. In a preferred embodiment, the polynucleotide is DNA. In some embodiments, the polynucleotide contains further nucleic acid sequences in addition to the sequence encoding the reverse transcriptase enzyme of the invention. In other embodiments, the polynucleotide consists of the sequence encoding the reverse transcriptase enzyme.

In further embodiments, the polynucleotide is codon-optimised for expression in a target host. Codon optimisation is a method by which gene expression and translational efficiency of a gene of interest is improved, ultimately, increasing protein expression in a target host, or host organism. The skilled person knows a plethora of codon optimisation approaches, for example using the most frequently used codon for all instances of an amino acid, adjusting codon usage to the natural distribution of the target host, or host organism, using codons that correspond to abundant tRNAs, replacing rate codons, avoiding codon pairs that are known for slow translation, or approaches that do not take these considerations in account which are also known as hypothesis-free approaches.

As laid out in the definition section above, a target host is any suitable organism or cellular subunit thereof. Therefore, a suitable organism can be for example, a yeast cell, a prokaryotic cell, or mammalian cells or cell lines that harbour the plasmid or polynucleotide comprising the reverse transcriptase according to the invention.

### Plasmids

In a further aspect, the invention provides a plasmid comprising the polynucleotide according to any of the preceding embodiments. Suitable plasmids comprising the inventive polynucleotide are for example cloning vectors and expression vectors, phage, phagemid, cosmid or any other nucleic acid that can be independently replicated in a suitable host cell, possesses at least one recognition sequence such as an endonuclease restriction site and into which another nucleic acid can be inserted. Suitable cloning and expression vectors for expressing the polynucleotide in a target host such as a prokaryotic cell, yeast cell or mammalian cells or cell lines are known to the skilled person.

### Cells

In a further aspect, the invention provides a cell comprising the reverse transcriptase according to any one of the preceding embodiments, the polynucleotide, or the plasmid of the preceding embodiments.

In preferred embodiments, the cell comprising the reverse transcriptase is a host cell. The host cell can be any cell that is suitable for expressing one or more recombinant proteins. Examples of suitable host cells include prokaryotic and eukaryotic cells. In one embodiment, the prokaryotic cell is for example, E. coli, Bacillus subtilis, Salmonella typhimurium, or species within the genera Pseudomonas, Streptomyces, and Staphylococcus. Suitable eukaryotic cells are for example animal cells, vertebrate cells, yeast cells, plant cells, fungal cells, insect cells, mammalian cells, reptile cells, or algae cells. In a preferred embodiment, the prokaryotic cell is E. coli, preferably E. coli JM109, E. coli JM101, E. coli XL-1 Blue, E. coli MV1 190, E. coli HB101, E. coli DH5α, E. coli DH10B, E. coli BL21.

The maintenance of host cells according to the invention is known to the skilled person. Similarly, the introduction of the reverse transcriptase according to any one of the preceding embodiments or the polynucleotide or the plasmid of the preceding embodiment to produce the respective protein is a routine procedure known to the skilled person.

### Methods

In another aspect, the invention provides a method for reverse transcription of RNA. Reverse transcription means that RNA is converted into cDNA. Therefore, in one embodiment, the reverse transcriptase according to any of the preceding embodiments is used to synthesise cDNA from RNA. In a preferred embodiment, the reverse transcriptase according to any of the preceding embodiments is used to synthesise cDNA from RNA in a sample.

In one embodiment, the sample is selected from the group consisting of a biological sample, environmental sample, or food product sample. In some embodiments, the biological samples, environmental samples, or food product samples can be fresh samples, frozen samples, or preserved samples, for example, preserved in formalin and/or paraffin-embedded. In further embodiments, the biological samples, environmental samples, or food product samples can contain further compounds such as metabolites of drugs, antibiotics, anticoagulants, chemicals such as preservatives, fixatives or buffers, nutrients, fertilisers or the like. In some embodiments, the biological samples, environmental samples, or food product samples are from a contaminated source.

In one embodiment, the biological sample is from a human being, an animal or a plant. In a preferred embodiment, the human being is a patient. In another preferred embodiment, the animal is a mammal. In a preferred embodiment, the biological sample is a cell, cell culture, blood, urine, faeces, saliva, cerebrospinal fluid, interstitial fluid, ascites, peritoneal fluid, amniotic fluid, breast milk, mucus, semen, bile, skin swab, or combinations thereof.

In another preferred embodiment, the environmental sample is a soil specimen, peat specimen, sediment specimen, or water specimen. In some embodiments, the water specimen may be a process water specimen, surface water specimen, potable water specimen, freshwater specimen, or seawater specimen. In yet another preferred embodiment, the environmental sample is a swab from a wall, floor, surface, ventilator, drain, conveyor belt, or container.

In yet another preferred embodiment, the food product sample is a sample of food or beverage. In some embodiments, the food product sample is a raw material, a material that is being processed, an “in-process sample”, or a sample from the finalised food or beverage product. In one embodiment, the raw material is raw vegetables, raw fruit, raw meat, spices, nuts, potable water, syrups, additives, preservatives, sugar, or artificial sweeteners. In another embodiment, the processed material is processed vegetables, processed fruit, processed meat, processed spices and processed nuts. In yet another embodiment, the finalised food product is a dairy product such as cheese or butter. In even another embodiment, the finalised beverage product is milk, lemonade, vegetable juice, fruit juice, or potable water.

In one embodiment, the biological sample, environmental sample, or food product sample is a processed sample or a purified sample.

In one embodiment, the processed sample is a sample that has been subjected to a defined process or treatment. Said defined process or treatment may comprise multiple steps. In some embodiments, the defined process or treatment may be for example, RNA or DNA extraction, reverse transcription, qPCR, RT-qPCR, PCR.

In some embodiments, a purified sample is subjected to a defined process or treatment, thereby becoming a processed sample. Hence, in some embodiments, a purified sample is a processed sample.

In another embodiment, a purified sample is a sample from which any substance that is potentially harmful, dirty or inferior has been removed. Therefore, in some embodiments, substances considered contaminants are removed from a purified sample. In another embodiment, a purified sample corresponds to a concentrated sample. In some embodiments, a purified sample is a processed sample. The skilled person in the art knows a range of chemical and physical purification methods such as filtration, drying, chromatography, distillation, extraction, ion exchange etc.

In another embodiment, the biological sample, environmental sample, or food product sample is an unprocessed sample or an unpurified sample.

In one embodiment, the unprocessed sample is a sample that has not been subjected to a defined process or treatment. In another embodiment, an unpurified sample is a sample from which any substance that is potentially harmful, dirty or inferior has not been removed. Therefore, in some embodiments, substances considered contaminants are present in an unpurified sample. In another embodiment, an unpurified sample corresponds to a non-concentrated sample.

### EXAMPLES

### Example 1: Single Point Mutants I416T (SEQ ID NO: 2), K267Q (SEQ ID NO: 3), N674D (SEQ ID NO: 4), and T186A (SEQ ID NO: 5) of Wild-Type MMLV Reverse Transcriptase (SEQ ID NO: 1)

In order to create a reverse transcriptase with improved inhibitor resistance, the four single point mutants I416T (SEQ ID NO: 2), K267Q (SEQ ID NO: 3) N674D (SEQ ID NO: 4), and T186A (SEQ ID NO: 5) were designed.

Mutations were introduced by site-directed mutagenesis with mutagenic primers using Invitrogen Platinum™ SuperFi™ II PCR Master Mix according to recommendations described in Application note, Mutagenesis protocol A: https://tools.thermofisher.com/content/sfs/brochures/site-directed-mutagenesis-platinum-superfi-app-note.pdf. Briefly, point mutations were created by designing two mutagenic primers, which are partially complementary with 3′-overhangs. PCR amplification was performed using Platinum™ SuperFi™ PCR Master Mix, followed by transformation of the PCR product without any additional steps in E. coli (pET21 expression system was used for protein expression), followed by purification. Subsequently, SDS-PAGE under reducing conditions was performed and purified variants yielded a single band with a molecular mass of 78 kDa; the achieved purity was more than 80%.

### Example 2: Inhibitor Resistance of Single Point Mutants I416T (SEQ ID NO: 2), K267Q (SEQ ID NO: 3) N674D (SEQ ID NO: 4), and T186A (SEQ ID NO: 5) Compared to Wild-Type MMLV Reverse Transcriptase (SEQ ID NO: 1)

The reaction mixture was prepared as laid out in Table 1 below:

TABLE 1
Reaction mixture

| Component | Final concentration | |
|---|---|---|
| 5X RT buffer | 1X | |
| dNTP Mix | 1 | mM |
| OligodT₁₈ | 5 | μM |
| RiboLock ™ RNase | 1 | U/μL |
| inhibitor | | |
| Reverse transcriptase | 10 | U/μL |
| Known RT inhibitor | Up to specific | |
| | concentration | |
| 1.3 kb GAPDH RNA | 50 | ng/μL |

| | |
|---|---|
| H₂O | to 20 μL |

5× RT buffer (second line of Table 1 above) consists of 250 mM Tris-HCl (pH 8.3 at 25° C.), 250 mM KCl, 20 mM MgCl₂, and 50 mM DTT.

Increasing amounts of the following inhibitors were added to the reaction mixtures, respectively: hemin (10 μM, 15 μM, 17 μM), tannic acid (5 ng/μL, 7.5 ng/μL, 10 ng/μL), humic acid (6 ng/μL, 8 ng/μL, 10 ng/μL), guanidine isothiocyanate (70 mM, 75 mM, 80 mM). Reaction mixtures were incubated at 42° C. for 60 minutes, subsequently mixed with 5 μL of alkaline electrophoresis dye (180 mM NaOH, 6 mM EDTA, 18% Ficoll™ 400, 0.05% bromocresol green), and heated at 70° C. for 10 minutes. Then, the samples were chilled on ice and loaded onto a 1.4% agarose gels as outlined in Baranauskas et al. (2012), Protein Engineering Design & Selection. The gel was prepared in 30 mM NaCl, 2 mM EDTA (pH 7.5) buffer and equilibrated in electrophoresis buffer (30 mM NaOH, 2 mM EDTA) at 4° C. overnight. Electrophoresis was performed at 3 V/cm for 1 hour, the gel was neutralized in 300 ml of 0.5 M Tris-HCl buffer (pH 7.5) for 30 minutes, stained in 0.5 μg/ml ethidium bromide solution for 30 minutes, followed by visualisation under UV light. All reactions were performed in three technical replicates.

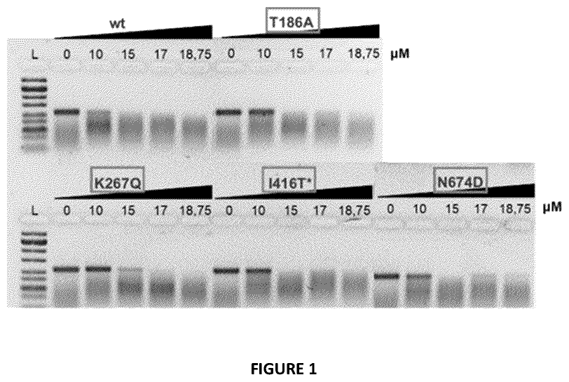

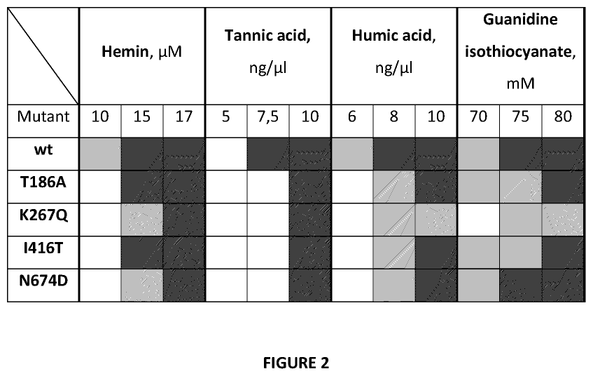

The results showed that the mutants T186A, K267Q I416T, and N674D were able to perform the synthesis of a 1.3 kb cDNA fragment in the presence of higher inhibitor concentrations than the wild-type MMLV reverse transcriptase, indicating their increased tolerance to the inhibitors. The results of cDNA synthesis in presence of hemin in the reaction mixtures are shown in FIG. 1. The results of cDNA synthesis in presence of a range of inhibitors, i.e. hemin, tannic acid, humic acid, and guanidine isothiocyanate are summarised and displayed in FIG. 2.

### Example 3: Increased Processivity of Single Point Mutant Reverse Transcriptases I416T (SEQ ID NO: 2), K267Q (SEQ ID NO: 3), N674D (SEQ ID NO: 4), and T186A (SEQ ID NO: 5) Compared to Wild-Type MMLV Reverse Transcriptase (SEQ ID NO: 1)

Synthesis of 0.5-9 kb cDNA fragments from Millennium™ RNA Markers (Invitrogen, #AM7150)

The reaction mixture composition was the same as provided in Table 2 except that 1 μg of Millennium™ RNA Markers was added to the mixture instead of GAPDH RNA.

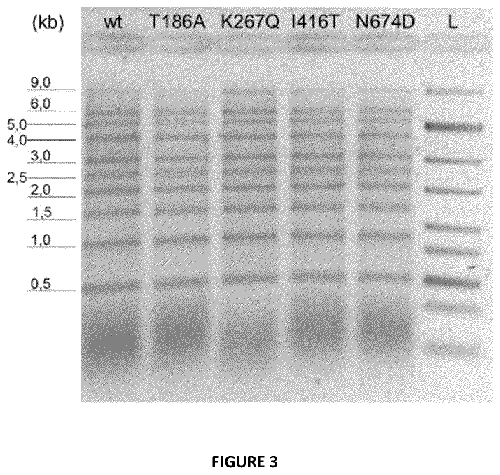

Increased processivity of single point mutants I416T (SEQ ID NO: 2), K267Q (SEQ ID NO: 3), N674D (SEQ ID NO: 4) and T186A (SEQ ID NO: 5) compared to wild-type MMLV reverse transcriptase (SEQ ID NO: 1), in particular of the K267Q mutant (SEQ ID NO: 3) has been demonstrated. The results are shown in FIG. 3.

### Example 4. Increased Thermostability and Thermoactivity of Single Point Mutant Reverse Transcriptase I416T (SEQ ID NO: 2) Compared to Wild-Type MMLV Reverse Transcriptase (SEQ ID NO: 1)

The reaction mixture composition was the same as provided in Table 1 except that 1 μg of Millennium™ RNA Markers was added to the mixture instead of GAPDH RNA.

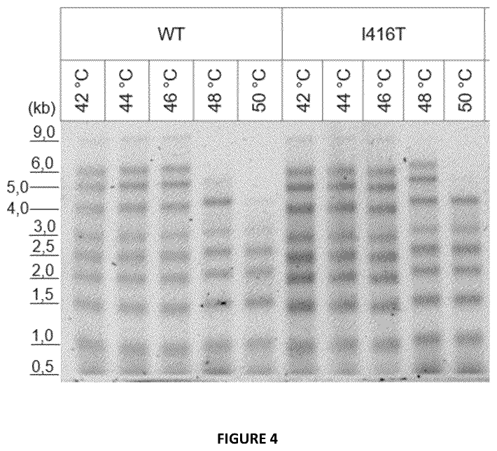

cDNA synthesis reactions were carried out at 42° C., 44° C., 46° C., 48° C., 50° C. for 60 minutes, subsequently mixed with 5 μL of alkaline electrophoresis buffer and heated at 70° C. for 10 minutes. Samples were analysed in 1.0% alkaline agarose gels as described in Example 2. The results of cDNA synthesis in temperature gradient are shown in FIG. 4.

The results showed that the mutant RT I416T was able to synthesize longer cDNA fragments than the wild-type MMLV reverse transcriptase at 48° C. and at 50° C., indicating its higher thermostability and thermoactivity.

### Example 5. Increased Thermostability of Single Point Mutant Reverse Transcriptase I416T (SEQ ID NO: 2) Compared to Wild-Type MMLV Reverse Transcriptase (SEQ ID NO: 1)

The reaction mixture was prepared as laid out in Table 2 below:

TABLE 2
Reaction mixture part 1

| Component | Quantity | |
|---|---|---|
| 5X RT buffer | 4 | μL |
| Reverse transcriptase | 200 | U |
| H₂O | 5.5 | μL |

Then, the mixture was incubated at 55° C. for 30 minutes. After incubation, the remaining components were added:

TABLE 3
Reaction mixture part 2

| Component | Final concentration | |
|---|---|---|
| Oligo dT₁₈ | 5 | μM |
| Millennium ™ RNA Markers | 50 | ng/μL |
| dNTP Mix | 1 | mM |
| RiboLock RNase inhibitor | 1 | U/μL |

| | |
|---|---|
| H₂O | to 20 μL |

cDNA synthesis reactions were carried out at 50° C. for 30 minutes and said reactions were stopped by heating at 80° C. for 10 minutes and mixed with 6× TrackIt™ Cyan/Orange Loading Buffer. 12 μL of each mixture was analysed on a 1% TAE agarose gel stained in 0.5 μg/ml ethidium bromide solution.

Single point mutant reverse transcriptase I416T (SEQ ID NO: 2) exhibited cDNA synthesis activity even after thermal treatment, while the wild-type MMLV reverse transcriptase almost lost its activity after incubation at 55° C. (data not shown).

TABLE 4
SEQUENCES
Sequences (SEQ ID NOs: 1-16)

| SEQ | | |
|---|---|---|
| ID | | Name of the |
| NO: | Sequence | sequence |
| 1 | TLNIEDEHRLHETSKEPDVSLGSTWLSDFPQAWAETGGMGLAVRQAPLIIPL | wild-type |
| | KATSTPVSIKQYPMSQEARLGIKPHIQRLLDQGILVPCQSPWNTPLLPVKKP | Moloney |
| | GTNDYRPVQDLREVNKRVEDIHPTVPNPYNLLSGLPPSHQWYTVLDLKDAFF | Murine |
| | CLRLHPTSQPLFAFEWRDPEMGISGQLTWTRLPQGFKNSPTLFDEALHRDLA | Leukaemia |
| | DFRIQHPDLILLQYVDDLLLAATSELDCQQGTRALLQTLGNLGYRASAKKAQ | Virus reverse |
| | ICQKQVKYLGYLLKEGQRWLTEARKETVMGQPTPKTPRQLREFLGTAGFCRL | transcriptase |
| | WIPGFAEMAAPLYPLTKTGTLENWGPDQQKAYQEIKQALLTAPALGLPDLTK | (MMLV RT) |
| | PFELFVDEKQGYAKGVLTQKLGPWRRPVAYLSKKLDPVAAGWPPCLRMVAAI | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIENSS | |
| | PNSRLIN | |
| 2 | TLNIEDEHRLHETSKEPDVSLGSTWLSDFPQAWAETGGMGLAVRQAPLIIPL | single mutant |
| | KATSTPVSIKQYPMSQEARLGIKPHIQRLLDQGILVPCQSPWNTPLLPVKKP | 1416T |
| | GTNDYRPVQDLREVNKRVEDIHPTVPNPYNLLSGLPPSHQWYTVLDLKDAFF | |
| | CLRLHPTSQPLFAFEWRDPEMGISGQLTWTRLPQGFKNSPTLFDEALHRDLA | |
| | DFRIQHPDLILLQYVDDLLLAATSELDCQQGTRALLQTLGNLGYRASAKKAQ | |
| | ICQKQVKYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAT | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIENSS | |
| | PNSRLIN | |
| 3 | TLNIEDEHRLHETSKEPDVSLGSTWLSDFPQAWAETGGMGLAVRQAPLIIPL | single mutant |
| | KATSTPVSIKQYPMSQEARLGIKPHIQRLLDQGILVPCQSPWNTPLLPVKKP | K267Q |
| | GTNDYRPVQDLREVNKRVEDIHPTVPNPYNLLSGLPPSHQWYTVLDLKDAFF | |
| | CLRLHPTSQPLFAFEWRDPEMGISGQLTWTRLPQGFKNSPTLFDEALHRDLA | |
| | DFRIQHPDLILLQYVDDLLLAATSELDCQQGTRALLQTLGNLGYRASAKKAQ | |
| | ICQKQVQYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAI | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIENSS | |
| | PNSRLIN | |
| 4 | TLNIEDEHRLHETSKEPDVSLGSTWLSDFPQAWAETGGMGLAVRQAPLIIPL | single mutant |
| | KATSTPVSIKQYPMSQEARLGIKPHIQRLLDQGILVPCQSPWNTPLLPVKKP | N674D |
| | GTNDYRPVQDLREVNKRVEDIHPTVPNPYNLLSGLPPSHQWYTVLDLKDAFF | |
| | CLRLHPTSQPLFAFEWRDPEMGISGQLTWTRLPQGFKNSPTLFDEALHRDLA | |
| | DFRIQHPDLILLQYVDDLLLAATSELDCQQGTRALLQTLGNLGYRASAKKAQ | |
| | ICQKQVKYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAI | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIEDSS | |
| | PNSRLIN | |
| 5 | TLNIEDEHRLHETSKEPDVSLGSTWLSDFPQAWAETGGMGLAVRQAPLIIPL | single mutant |
| | KATSTPVSIKQYPMSQEARLGIKPHIQRLLDQGILVPCQSPWNTPLLPVKKP | T186A |
| | GTNDYRPVQDLREVNKRVEDIHPTVPNPYNLLSGLPPSHQWYTVLDLKDAFF | |
| | CLRLHPTSQPLFAFEWRDPEMGISGQLTWARLPQGFKNSPTLFDEALHRDLA | |
| | DFRIQHPDLILLQYVDDLLLAATSELDCQQGTRALLQTLGNLGYRASAKKAQ | |
| | ICQKQVKYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAI | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIENSS | |
| | PNSRLIN | |
| 6 | TLNIEDEHRLHETSKEPDVSLGSTWLSDFPQAWAETGGMGLAVRQAPLIIPL | double mutant |
| | KATSTPVSIKQYPMSQEARLGIKPHIQRLLDQGILVPCQSPWNTPLLPVKKP | 1416T and |
| | GTNDYRPVQDLREVNKRVEDIHPTVPNPYNLLSGLPPSHQWYTVLDLKDAFF | K267Q |
| | CLRLHPTSQPLFAFEWRDPEMGISGQLTWTRLPQGFKNSPTLFDEALHRDLA | |
| | DFRIQHPDLILLQYVDDLLLAATSELDCQQGTRALLQTLGNLGYRASAKKAQ | |
| | ICQKQVQYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAT | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIENSS | |
| | PNSRLIN | |
| 7 | TLNIEDEHRLHETSKEPDVSLGSTWLSDFPQAWAETGGMGLAVRQAPLIIPL | Double mutant |
| | KATSTPVSIKQYPMSQEARLGIKPHIQRLLDQGILVPCQSPWNTPLLPVKKP | 1416T and |
| | GTNDYRPVQDLREVNKRVEDIHPTVPNPYNLLSGLPPSHQWYTVLDLKDAFF | N674D |
| | CLRLHPTSQPLFAFEWRDPEMGISGQLTWTRLPQGFKNSPTLFDEALHRDLA | |
| | DFRIQHPDLILLQYVDDLLLAATSELDCQQGTRALLQTLGNLGYRASAKKAQ | |
| | ICQKQVKYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAT | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIEDSS | |
| | PNSRLIN | |
| 8 | TLNIEDEHRLHETSKEPDVSLGSTWLSDFPQAWAETGGMGLAVRQAPLIIPL | Double mutant |
| | KATSTPVSIKQYPMSQEARLGIKPHIQRLLDQGILVPCQSPWNTPLLPVKKP | 1416T and |
| | GTNDYRPVQDLREVNKRVEDIHPTVPNPYNLLSGLPPSHQWYTVLDLKDAFF | T186A |
| | CLRLHPTSQPLFAFEWRDPEMGISGQLTWARLPQGFKNSPTLFDEALHRDLA | |
| | DFRIQHPDLILLQYVDDLLLAATSELDCQQGTRALLQTLGNLGYRASAKKAQ | |
| | ICQKQVKYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAT | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIENSS | |
| | PNSRLIN | |
| 9 | TLNIEDEHRLHETSKEPDVSLGSTWLSDFPQAWAETGGMGLAVRQAPLIIPL | Double mutant |
| | KATSTPVSIKQYPMSQEARLGIKPHIQRLLDQGILVPCQSPWNTPLLPVKKP | K267Q and |
| | GTNDYRPVQDLREVNKRVEDIHPTVPNPYNLLSGLPPSHQWYTVLDLKDAFF | N674D |
| | CLRLHPTSQPLFAFEWRDPEMGISGQLTWTRLPQGFKNSPTLFDEALHRDLA | |
| | DFRIQHPDLILLQYVDDLLLAATSELDCQQGTRALLQTLGNLGYRASAKKAQ | |
| | ICQKQVQYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAI | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIEDSS | |
| | PNSRLIN | |
| 10 | TLNIEDEHRLHETSKEPDVSLGSTWLSDFPQAWAETGGMGLAVRQAPLIIPL | Double mutant |
| | KATSTPVSIKQYPMSQEARLGIKPHIQRLLDQGILVPCQSPWNTPLLPVKKP | K267Q and |
| | GTNDYRPVQDLREVNKRVEDIHPTVPNPYNLLSGLPPSHQWYTVLDLKDAFF | T186A |
| | CLRLHPTSQPLFAFEWRDPEMGISGQLTWARLPQGFKNSPTLFDEALHRDLA | |
| | DFRIQHPDLILLQYVDDLLLAATSELDCQQGTRALLQTLGNLGYRASAKKAQ | |
| | ICQKQVQYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAI | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIENSS | |
| | PNSRLIN | |
| 11 | TLNIEDEHRLHETSKEPDVSLGSTWLSDFPQAWAETGGMGLAVRQAPLIIPL | Double mutant |
| | KATSTPVSIKQYPMSQEARLGIKPHIQRLLDQGILVPCQSPWNTPLLPVKKP | N674D and |
| | GTNDYRPVQDLREVNKRVEDIHPTVPNPYNLLSGLPPSHQWYTVLDLKDAFF | T186A |
| | CLRLHPTSQPLFAFEWRDPEMGISGQLTWARLPQGFKNSPTLFDEALHRDLA | |
| | DFRIQHPDLILLQYVDDLLLAATSELDCQQGTRALLQTLGNLGYRASAKKAQ | |
| | ICQKQVKYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAI | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIEDSS | |
| | PNSRLIN | |
| 12 | TLNIEDEHRLHETSKEPDVSLGSTWLSDFPQAWAETGGMGLAVRQAPLIIPL | Triple mutant |
| | KATSTPVSIKQYPMSQEARLGIKPHIQRLLDQGILVPCQSPWNTPLLPVKKP | 1416T, K267Q |
| | GTNDYRPVQDLREVNKRVEDIHPTVPNPYNLLSGLPPSHQWYTVLDLKDAFF | and N674D |
| | CLRLHPTSQPLFAFEWRDPEMGISGQLTWTRLPQGFKNSPTLFDEALHRDLA | |
| | DFRIQHPDLILLQYVDDLLLAATSELDCQQGTRALLQTLGNLGYRASAKKAQ | |
| | ICQKQVQYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAT | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIEDSS | |
| | PNSRLIN | |
| 13 | TLNIEDEHRLHETSKEPDVSLGSTWLSDFPQAWAETGGMGLAVRQAPLIIPL | Triple mutant |
| | KATSTPVSIKQYPMSQEARLGIKPHIQRLLDQGILVPCQSPWNTPLLPVKKP | 1416T, K267Q |
| | GTNDYRPVQDLREVNKRVEDIHPTVPNPYNLLSGLPPSHQWYTVLDLKDAFF | and T186A |
| | CLRLHPTSQPLFAFEWRDPEMGISGQLTWARLPQGFKNSPTLFDEALHRDLA | |
| | DFRIQHPDLILLQYVDDLLLAATSELDCQQGTRALLQTLGNLGYRASAKKAQ | |
| | ICQKQVQYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAT | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIENSS | |
| | PNSRLIN | |
| 14 | TLNIEDEHRLHETSKEPDVSLGSTWLSDFPQAWAETGGMGLAVRQAPLIIPL | Triple mutant |
| | KATSTPVSIKQYPMSQEARLGIKPHIQRLLDQGILVPCQSPWNTPLLPVKKP | 1416T, N674D |
| | GTNDYRPVQDLREVNKRVEDIHPTVPNPYNLLSGLPPSHQWYTVLDLKDAFF | and T186A |
| | CLRLHPTSQPLFAFEWRDPEMGISGQLTWARLPQGFKNSPTLFDEALHRDLA | |
| | DFRIQHPDLILLQYVDDLLLAATSELDCQQGTRALLQTLGNLGYRASAKKAQ | |
| | ICQKQVKYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAT | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIEDSS | |
| | PNSRLIN | |
| 15 | TLNIEDEHRLHETSKEPDVSLGSTWLSDFPQAWAETGGMGLAVRQAPLIIPL | Triple mutant |
| | KATSTPVSIKQYPMSQEARLGIKPHIQRLLDQGILVPCQSPWNTPLLPVKKP | K267Q, N674D |
| | GTNDYRPVQDLREVNKRVEDIHPTVPNPYNLLSGLPPSHQWYTVLDLKDAFF | and T186A |
| | CLRLHPTSQPLFAFEWRDPEMGISGQLTWARLPQGFKNSPTLFDEALHRDLA | |
| | DFRIQHPDLILLQYVDDLLLAATSELDCQQGTRALLQTLGNLGYRASAKKAQ | |
| | ICQKQVQYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAI | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIEDSS | |
| | PNSRLIN | |
| 16 | TLNIEDEHRLHETSKEPDVSLGSTWLSDFPQAWAETGGMGLAVRQAPLIIPL | Quadruple |
| | KATSTPVSIKQYPMSQEARLGIKPHIQRLLDQGILVPCQSPWNTPLLPVKKP | mutant 1416T, |
| | GTNDYRPVQDLREVNKRVEDIHPTVPNPYNLLSGLPPSHQWYTVLDLKDAFF | K267Q, N674D |
| | CLRLHPTSQPLFAFEWRDPEMGISGQLTWARLPQGFKNSPTLFDEALHRDLA | and T186A |
| | DERIQHPDLILLQYVDDLLLAATSELDCQQGTRALLQTLGNLGYRASAKKAQ | |
| | ICQKQVQYLGYLLKEGQRWLTEARKETVMGQPTPKTPRQLREFLGTAGFCRL | |
| | WIPGFAEMAAPLYPLTKTGTLFNWGPDQQKAYQEIKQALLTAPALGLPDLTK | |
| | PFELFVDEKQGYAKGVLTQKLGPWRRPVAYLSKKLDPVAAGWPPCLRMVAAT | |
| | AVLTKDAGKLTMGQPLVILAPHAVEALVKQPPDRWLSNARMTHYQALLLDTD | |
| | RVQFGPVVALNPATLLPLPEEGLQHNCLDILAEAHGTRPDLTDQPLPDADHT | |
| | WYTDGSSLLQEGQRKAGAAVTTETEVIWAKALPAGTSAQRAELIALTQALKM | |
| | AEGKKLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLIEDSS | |
| | PNSRLIN | |

### REFERENCES

BARANAUSKAS, A. Generation and characterization of new highly thermostable and processive M-MuLV reverse transcriptase variants. Protein Engineering, Design and Selection, October 2012, Vol. 25, No. 10, pages 657-668.
CAROTHERS, A M et al. Point mutation analysis in a mammalian gene: Rapid preparation of total RNA, PCR amplification of cDNA, and Taq sequencing by a novel method. Biotechniques, May 1989, Vol. 7, No. 5, pages 494-499.
KRISHNAN, B R et al. Linear amplification DNA sequencing directly from single phage plaques and bacterial colonies. Nucleic Acid Research, March 1991, Vol. 19, No. 5, page 1153.
CLARK, J M. Novel non-templated nucleotide addition reactions catalyzed by procaryotic and eucaryotic DNA polymerases. Nucleic Acid Research, October 1988, Vol. 16, No. 20, pages 9677-9686.
LIN, Y AND JAYASENA, S D. Inhibition of multiple thermostable DNA polymerases by a heterodimeric aptamer. Journal of Molecular Biology, August 1997, Vol. 264, Issue 1, pages 100-111.
DANG, C AND JAYESENA, S D. Oligonucleotide inhibitors of Taq DNA polymerase facilitate detection of low copy number targets by PCR. Journal of Molecular Biology, November 1996, Vol. 271, Issue 2, pages 268-278.
SCALICE, E R et al. Monoclonal antibodies prepared against the DNA polymerase from Thermus aquaticus are potent inhibitors of enzyme activity. Journal of Immunological Methods, June 1994, Vol. 172, Issue 2, pages 147-163.
SHARKEY, D et al. Antibodies as Thermolabile Switches: High Temperature Triggering for the Polymerase Chain Reaction. Nature Biotechnology, Vol. 12, pages 506-509.
MORETTI, T. et al. Enhancement of PCR Amplification Yield and Specificity Using AmpliTaq Gold™ DNA Polymerase. BioTechniques, October 1998, Vol. 25, pages 716-722

1. A reverse transcriptase enzyme comprising a modified Moloney Murine Leukaemia Virus reverse transcriptase amino acid sequence, wherein said amino acid sequence comprises a mutation at position K267, and/or I416, and/or N674, and/or T186 compared to the wild-type Moloney Murine Leukaemia Virus reverse transcriptase of SEQ ID NO: 1, wherein said enzyme possesses increased inhibitor resistance compared to said wild-type Moloney Murine Leukaemia Virus reverse transcriptase.

2. The reverse transcriptase enzyme according to claim 1, wherein said amino acid sequence has at least two mutations at positions selected from the group consisting of: K267, I416, N674 and T186.

3-4. (canceled)

5. The reverse transcriptase enzyme according to claim 1, wherein the amino acid sequence comprises at least one further mutation at one or more of the following amino acid positions:

| | | | | |
|---|---|---|---|---|
| P51 | H204 | N249 | T306 | F309 |
| D524 | E562 | K571 | D583 | |
| T330 | N479 | L603 | M289 | |
| M66 | L139 | D200 | T287 | H594 |
| E607 | Q221 | I49 | A502 | D653 |
| K658 | P130 | Q237 | A307 | Y344 |
| Q430 | D449 | A644 | N649 | L671 |
| E673 | M39 | Q91 | W388 | I179 |
| L333 | R390 | Q374 | E5 | |
| E69 | E302 | W313 | L435 | N454 |
| D124 | E286 | | | |

6. The reverse transcriptase enzyme according to claim 1, comprising a mutation selected from the group consisting of I416Q, I416N, I416T, I416S.

7. The reverse transcriptase enzyme according to claim 6, comprising an I416T mutation (SEQ ID NO: 2).

8. The reverse transcriptase enzyme according to claim 1, comprising a mutation selected from the group consisting of K267Q, K267N, K267S.

9. The reverse transcriptase enzyme according to claim 8, comprising a K267Q mutation (SEQ ID NO: 3).

10. The reverse transcriptase enzyme according to claim 1, comprising a mutation selected from the group consisting of N674D or N674E.

11. The reverse transcriptase enzyme according to claim 10, comprising a N674D mutation (SEQ ID NO: 4).

12. The reverse transcriptase enzyme according to claim 1, comprising:
i. a mutation at position K267 that is not K267T; or
ii. a mutation at position 186 that is not T186C or T186D.

13. (canceled)

14. The reverse transcriptase enzyme according to claim 12, comprising a mutation selected from the group consisting of T186A, T186V, T186I, T186L, T186M.

15. The reverse transcriptase enzyme according to claim 14, comprising a T186A mutation (SEQ ID NO: 5).

16. The reverse transcriptase enzyme according to claim 1, comprising an I416T mutation, a K267Q mutation, a N674D mutation, and/or a T186A mutation.

17-18. (canceled)

19. The reverse transcriptase enzyme according to claim 1, wherein said enzyme confers increased resistance to hemin, tannic acid, humic acid, and guanidine isothiocyanate.

20. A composition comprising the reverse transcriptase according to claim 1.

21-22. (canceled)

23. A kit comprising the reverse transcriptase according to claim 1.

24. A polynucleotide encoding the reverse transcriptase enzyme according to claim 1.

25. (canceled)

26. A plasmid or host cell comprising the polynucleotide of claim 24.

27. (canceled)

28. A method for reverse transcription of RNA, wherein the reverse transcriptase according to claim 1 is used to synthesise cDNA from RNA in a sample.

29. The method of claim 28, wherein the sample is selected from the group consisting of a biological sample, environmental sample, and food product sample.

30-31. (canceled)
